(19) United States
(12) Patent Application Publication
(10) Pub. No.: US 2015/0250786 A1
(43) Pub. Date: Sep. 10, 2015

(54) PYRIMIDINE HYDROXY AMIDE COMPOUNDS AS PROTEIN DEACETYLASE INHIBITORS AND METHODS OF USE THEREOF

(71) Applicant: THE TRUSTEES OF THE UNIVERSITY OF PENNSYLVANIA, Philadelphia, PA (US)

(72) Inventor: Olivier Roland Berton, Philadelphia, PA (US)

(73) Assignee: THE TRUSTEES OF THE UNIVERSITY OF PENNSYLVANIA, Philadelphia, PA (US)

(21) Appl. No.: 14/435,027

(22) PCT Filed: Oct. 11, 2013
(86) PCT No.: PCT/US13/64593

§ 371 Date: Apr. 10, 2015

Related U.S. Application Data
(60) Provisional application No. 61/713,014, filed on Oct. 12, 2012.

(51) Int. Cl. A61K 31/505

(52) U.S. Cl. CPC A61K 31/505

(57) ABSTRACT

The present invention relates to pyrimidine hydroxy amide compounds, and the use of such compounds in the inhibition of HDAC6 and in the treatment of depression and/or anxiety.

### CROSS-REFERENCE TO RELATED APPLICATIONS

This application claims priority to U.S. Provisional Application No. 61/713,014, filed Oct. 12, 2012, the content of which is incorporated herein by reference in its entirety.

### STATEMENT REGARDING FEDERALLY SPONSORED RESEARCH

Some research conducted for this invention was funded by the US Federal government (grant MH087581 from the U.S. National Institute of Mental Health, NIH) and by awards from the International Mental Health Research Organization (IMHRO) and NARSAD.

### BACKGROUND OF THE INVENTION

A biological target of recent interest is histone deacetylase (HDAC). Post-translational modification of proteins through acetylation and deacetylation of lysine residues plays a critical role in regulating their cellular functions. HDACs are zinc hydrolases that modulate gene expression through deacetylation of the N-acetyl-lysine residues of histone proteins and other transcriptional regulators (Hassig et al Curr. Opin. Chem. Biol. 1997, 1, 300-308). HDACs participate in cellular pathways that control cell shape and differentiation. At this time, eleven human HDACs, which use Zn as a cofactor, have been identified (Taunton et al. Science 1996, 272, 408-411; Yang et al. J. Biol. Chem. 1997, 272, 28001-28007. Grozinger et al. Proc. Natl. Acad. Sci. U.S.A. 1999, 96, 4868-4873; Kao et al. Genes Dev. 2000, 14, 55-66. Hu et al J. Biol. Chem. 2000, 275, 15254-15264; Zhou et al. Proc. Natl. Acad. Sci. U.S.A. 2001, 98, 10572-10577; Venter et al. Science 2001, 291, 1304-1351) these members fall into three classes (class I, II, and IV). An additional seven HDACs have been identified which use NAD as a cofactor. To date, no small molecules are known that selectively target any particular class or individual members of this family (for example ortholog-selective HDAC inhibitors have been reported: (a) Meinke et al. J. Med. Chem. 2000, 14, 4919-4922; (b) Meinke, et al Curr. Med. Chem. 2001, 8, 211-235). There remains a need for preparing structurally diverse HDAC and tubulin deacetylase (TDAC) inhibitors particularly ones that are potent and/or selective inhibitors of particular classes of HDACs or TDACs and individual HDACs and TDACs.

Histone deacetylases are a family of at least 11 zinc-binding hydrolases, which catalyze the deacetylation of lysine residues on histone proteins. HDAC inhibition results in hyperacetylation of chromatin, alterations in transcription, and growth arrest. Until recently selective HDAC inhibitors have not been realized.

HDAC6 is believed to bind ubiquitinated proteins through a zinc finger domain and interacts with the dynein motor complex through another discrete binding motif. HDAC6 possesses two catalytic deacetylase domains.

Provided herein are small molecule inhibitors of HDAC6, pharmaceutical compositions thereof, and methods of using these compounds to treat depression, anxiety, or both depression and anxiety. In certain embodiments, these compounds are potent and selective inhibitors of HDAC6. In certain embodiments, these compounds penetrate the blood-brain barrier.

### SUMMARY OF THE INVENTION

The invention provides a method for treating depression and/or anxiety in a subject comprising administering to the subject a compound of formula I.

In another aspect, the invention provides a method of treating a subject suffering from or susceptible to depression and/or anxiety comprising administering to a subject in need thereof a therapeutically effective amount of a compound of formula I, to thereby treat the subject suffering from or susceptible to depression and/or anxiety.

In an embodiment, the depression to be treated is one or more of the following: major depression, clinical depression, chronic depression, dysthymia, atypical depression, bipolar depression, manic depression, seasonal depression, phychotic depression, and postpartum depression.

In an embodiment, the anxiety to be treated is one or more of the following: generalized anxiety disorder, obsessive-compulsive disorder (OCD), panic disorder, post-traumatic stress disorder (PTSD), and social phobia (or social anxiety disorder).

In accordance with these methods, provided herein is a compound of formula I:

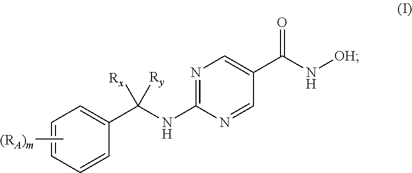

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and
m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

In another aspect, the invention provides a pharmaceutical composition comprising a compound of formula I.

In another aspect, the invention provides a method of selectively inhibiting HDAC6 over other HDACs in a subject, comprising administering to the subject a compound of formula I. In a non-limiting embodiment, this selective inhibition results in the treatment of depression. In another embodiment, this selective inhibition results in the treatment of anxiety. In a further embodiment, this selective inhibition results in the treatment of both depression and anxiety.

In another aspect, the invention provides a kit comprising a compound capable of inhibiting HDAC activity selected from one or more compounds of formula I; and instructions for use in treating depression, anxiety, or both depression and anxiety.

### BRIEF DESCRIPTION OF THE DRAWINGS

FIG. 1 shows tubulin acetylation changes after acute intraperitoneal (i.p.) injection with Tubastatin A, Compound 73, or Compound 101 at 30 minutes, 1 hour, and four hours post-injection.

FIG. 2 shows tubulin acetylation in serotonin rat neuroblastoma (RN46A) cells after treatment with TSA, Tubastatin A, Compound 73, or Compound 101 at 1 hour, 4 hours, and 24 hours post-injection.

FIG. 3 shows a comparison of tubulin acetylation in chronic versus subchronic drug treatment using either Compound 73 or Compound 101 in various cerebral tissues.

FIG. 4 shows histone 3 acetylation after treatment with Compound 73 at 30 minutes and 4 hours after treatment.

FIG. 5 shows the percent change in tubulin acetylation after subchronic drug treatment with Compound 73 or Compound 101 in HDAC6 knockout and wild-type animals.

FIG. 6 shows open field locomotor activity after an acute dose of Compound 73 at 30 minutes, 1 hour, 1.5 hours, and 2 hours after treatment.

FIG. 7 shows open field locomotor activity after an acute dose of Compound 101 at 30 minutes, 1 hour, 1.5 hours, and 2 hours after treatment.

FIG. 8 shows total locomotor activity over 2 hours after treatment with Compound 73 or Compound 101.

FIG. 9 shows social interaction after 20 days of chronic inhibitor treatment withFluoxetine, Compound 73, or Compound 101, and 10 days of social defeat.

FIG. 10 shows the results of a tail suspension test using a combination of selective serotonin reuptake inhibitor (SSRI) and histone deacetylase 6 (HDAC6) inhibition.

FIG. 11 shows the results of a tail suspension test with acute treatment with histone deacytelase (HDAC6) inhibitors.

FIG. 12 shows the plasma and brain levels of Compound 101 one hour following a 5 mg/kg intraperitoneal (ip) dose in mice.

FIGS. 13a, 13b, 13c, 13d, 13e, and 13f show the selectivity, potency and pharmacokinetic properties of the HDAC6 inhibitors used in present study.

FIGS. 14a, 14b, 14c, and 14d show the effect of HDAC6 inhibitors on α-tubulin acetylation at lysine 40 (K40) and Histone H3 acetylation at lysine 9 (H3K9) in neuronal cell culture (14a) and in the CNS in vivo after acute and subchronic administration (14b-c). * p<0.05, ** p<0.01, ***p<0.001 versus vehicle (n=2-3 per condition).

FIGS. 15a, 15b, 15c, 15d, 15e and 15f show the effect of HDAC6 selective inhibition in anxiety tests. Data are expressed as mean values±SEM. * p<0.05, ** p<0.01, ***p<0.001 versus the vehicle.

FIGS. 16a, 16b, 16c, 16d, 16e, 16f and 16g show that the HDAC6 inhibitors compound 73 and compound 101 have antidepressant-like properties. Data was expressed as mean values±SEM.+p<0.05 versus vehicle, # p<0.01 versus vehicle, * p<0.01 versus undefeated control.

### DETAILED DESCRIPTION OF THE INVENTION

### Definitions

Listed below are definitions of various terms used to describe this invention. These definitions apply to the terms as they are used throughout this specification and claims, unless otherwise limited in specific instances, either individually or as part of a larger group.

The number of carbon atoms in a hydrocarbyl substituent can be indicated by the prefix “Cₓ-C_{y},” where x is the minimum and y is the maximum number of carbon atoms in the substituent. Likewise, a Cₓ chain means a hydrocarbyl chain containing x carbon atoms.

If a linking element in a depicted structure is “absent” or a “bond”, then the left element in the depicted structure is directly linked to the right element in the depicted structure. For example, if a chemical structure is depicted as X-(L)ₙ-Y wherein L is absent or n is 0, then the chemical structure is X—Y.

The term “alkyl,” as used herein, refers to saturated, straight- or branched-chain hydrocarbon moieties containing, in certain embodiments, between one and six, or one and eight carbon atoms, respectively. Examples of C₁-C₆ alkyl moieties include, but are not limited to, methyl, ethyl, propyl, isopropyl, n-butyl, tert-butyl, neopentyl, n-hexyl moieties; and examples of C₁-C₈ alkyl moieties include, but are not limited to, methyl, ethyl, propyl, isopropyl, n-butyl, tert-butyl, neopentyl, n-hexyl, heptyl, and octyl moieties.

The term “alkenyl,” as used herein, denotes a monovalent group derived from a hydrocarbon moiety containing, in certain embodiments, from two to six, or two to eight carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “alkynyl,” as used herein, denotes a monovalent group derived from a hydrocarbon moiety containing, in certain embodiments, from two to six, or two to eight carbon atoms having at least one carbon-carbon triple bond. The alkynyl group may or may not be the point of attachment to another group. Representative alkynyl groups include, but are not limited to, for example, ethynyl, 1-propynyl, 1-butynyl, heptynyl, octynyl and the like.

The term “alkoxy” refers to an —O-alkyl moiety.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like.

The term “aralkyl,” or “arylalkyl,” as used herein, refers to an alkyl residue attached to an aryl ring. Examples include, but are not limited to, benzyl, phenethyl and the like.

The term “carbocyclic,” as used herein, denotes a monovalent group derived from a monocyclic or polycyclic saturated, partially unsaturated, or fully unsaturated carbocyclic ring compound. Examples of carbocyclic groups include groups found in the cycloalkyl definition and aryl definition.

The term “cycloalkyl,” as used herein, denotes a monovalent group derived from a monocyclic or polycyclic saturated or partially unsaturated carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl. Also contemplated are monovalent groups derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, moieties or ring system having at least one aromatic ring, having from five to ten ring atoms of which one ring atom is selected from S, O and N; zero, one or two ring atoms are additional heteroatoms independently selected from S, O and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl,” as used herein, refers to an alkyl residue residue attached to a heteroaryl ring. Examples include, but are not limited to, pyridinylmethyl, pyrimidinylethyl and the like.

The term “heterocycloalkyl,” as used herein, refers to a non-aromatic 3-, 4-, 5-, 6- or 7-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “alkylamino” refers to a group having the structure —NH(C₁-C₁₂ alkyl) where C₁-C₁₂ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

In accordance with the invention, any of the aryls, substituted aryls, heteroaryls and substituted heteroaryls described herein, can be any aromatic group. Aromatic groups can be substituted or unsubstituted.

The terms “hal,” “halo” and “halogen,” as used herein, refer to an atom selected from fluorine, chlorine, bromine and iodine.

The term “oxo” as used herein, refers to an oxygen that is attached to a carbon, preferably by a double bond (e.g., carbonyl).

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to:

alkyl, alkenyl, alkynyl, cycloalkyl, aryl, heterocycloalkyl, heteroaryl, arylalkyl, heteroarylalkyl, haloalkyl (e.g., —CF₃), haloalkoxy (e.g., —OCF₃),

—F, —Cl, —Br, —I,

—OH, protected hydroxy, oxygen, oxo,

—NO₂, —CN,

—NH₂, protected amino, —NH—C₁-C₁₂-alkyl, —NH-aryl, -dialkylamino, —

—O—C₁-C₁₂-alkyl, —O-aryl,

—C(O)—, —C(O)O—, —C(O)NH—, —OC(O)—, —OC(O)O—, —OC(O)NH—, —NHC(O)—, —NHC(O)O—,

—C(O)—C₁-C₁₂-alkyl, —C(O)—C₃-C₁₂-cycloalkyl, —C(O)-aryl, —C(O)-heteroaryl, —C(O)-heterocycloalkyl,

—C(O)O—C₁-C₁₂-alkyl, —C(O)O—C₃-C₁₂-cycloalkyl, —C(O)O-aryl, —C(O)O-heteroaryl, —C(O)O-heterocycloalkyl,

—CONH₂, —CONH—C₁-C₁₂-alkyl, —CONH-aryl,

—OCO₂—C₁-C₁₂-alkyl, —OCO₂-aryl, —OCONH₂, —OCONH—C₁-C₁₂-alkyl, —OCONH-aryl,

—NHC(O)— C₁-C₁₂-alkyl, —NHC(O)-aryl, —NHCO₂—C₁-C₁₂-alkyl, —NHCO₂-aryl,

—S(O)—C₁-C₁₂-alkyl, —S(O)-aryl, —SO₂NH—C₁-C₁₂-alkyl, —SO₂NH-aryl,

—NHSO₂—C₁-C₁₂-alkyl, —NHSO₂-aryl, or

—SH, —S—C₁-C₁₂-alkyl, or —S-aryl.

In certain embodiments, the optionally substituted groups include the following: C₁-C₁₂-alkyl, C₂-C₁₂-alkenyl, C₂-C₁₂-alkynyl, C₃-C₁₂-cycloalkyl, C₃-C₁₂-aryl, C₃-C₁₂-heterocycloalkyl, C₃-C₁₂-heteroaryl, C₄-C₁₂-arylalkyl, or C₂-C₁₂-heteroarylalkyl.

It is understood that the aryls, heteroaryls, alkyls, and the like can be further substituted.

As used herein, the term “metal chelator” refers to any molecule or moiety that is capable of forming a complex (i.e., “chelates”) with a metal ion. In certain exemplary embodiments, a metal chelator refers to to any molecule or moiety that “binds” to a metal ion, in solution, making it unavailable for use in chemical/enzymatic reactions. In certain embodiments, the solution comprises aqueous environments under physiological conditions.

Examples of metal ions include, but are not limited to, Ca²⁺, Fe³⁺, Zn²⁺, Na⁺, etc. In certain embodiments, the metal chelator binds Zn²⁺. In certain embodiments, molecules of moieties that precipitate metal ions are not considered to be metal chelators.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

“Treat”, “treating” and “treatment” refer to a method of alleviating or abating a disease and/or its attendant symptoms.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug,” as used herein means a compound which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating disorders through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxyysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

As used herein, the term “disease” includes depression, major depression, clinical depression, chronic depression, dysthymia, atypical depression, bipolar depression, manic depression, seasonal depression, phychotic depression, postpartum depression, anxiety, generalized anxiety disorder, obsessive-compulsive disorder (OCD), panic disorder, post-traumatic stress disorder (PTSD), and social phobia (or social anxiety disorder).

Compounds of the Invention

Provided herein is a compound of formula I:

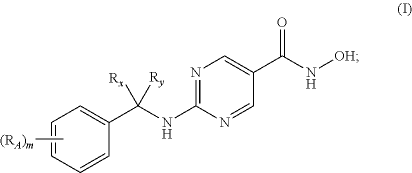

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

Representative compounds of Formula I include, but are not limited to, the following compounds of Table 1 below, or pharmaceutically acceptable salts, esters or prodrugs thereof.

TABLE 1

| | |
|---|---|
| 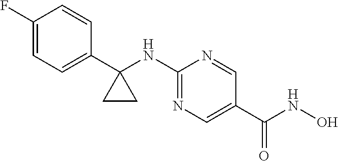 [SMILES: O=C(NO)c1cnc(NC2(c3ccc(F)cc3)CC2)nc1] | 81 |
| 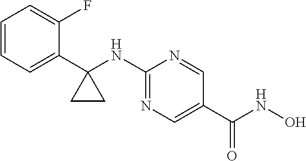 [SMILES: O=C(NO)c1cnc(NC2(c3ccccc3F)CC2)nc1] | 82 |
| 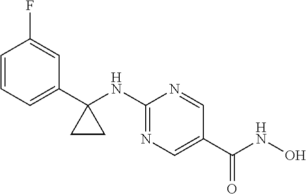 [SMILES: O=C(NO)c1cnc(NC2(c3cccc(F)c3)CC2)nc1] | 87 |
| 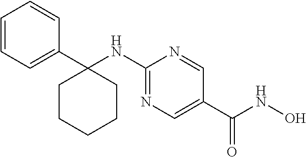 [SMILES: O=C(NO)c1cnc(NC2(c3ccccc3)CCCCC2)nc1] | 84 |
| 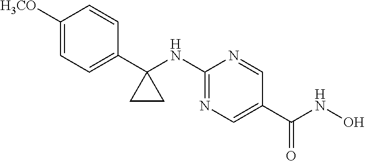 [SMILES: COc1ccc(C2(Nc3ncc(C(=O)NO)cn3)CC2)cc1] | 97 |
| 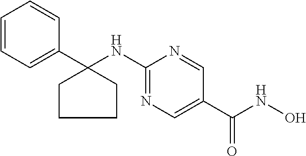 [SMILES: O=C(NO)c1cnc(NC2(c3ccccc3)CCCC2)nc1] | 88 |
| 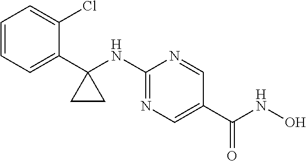 [SMILES: O=C(NO)c1cnc(NC2(c3ccccc3Cl)CC2)nc1] | 90 |
| 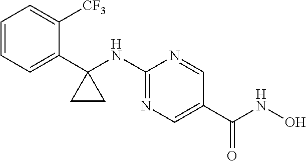 [SMILES: O=C(NO)c1cnc(NC2(c3ccccc3C(F)(F)F)CC2)nc1] | 91 |
| 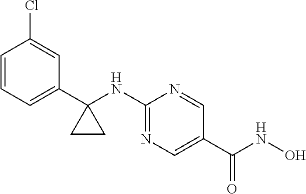 [SMILES: O=C(NO)c1cnc(NC2(c3cccc(Cl)c3)CC2)nc1] | 94 |
| 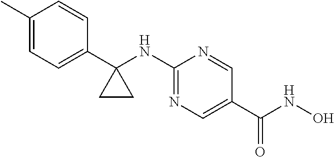 [SMILES: Cc1ccc(C2(Nc3ncc(C(=O)NO)cn3)CC2)cc1] | 93 |
| 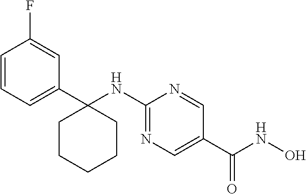 [SMILES: O=C(NO)c1cnc(NC2(c3cccc(F)c3)CCCCC2)nc1] | 101 |
| (ACY-775) | |
| 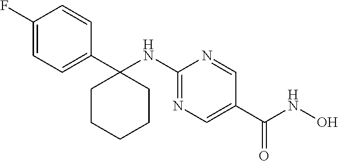 [SMILES: O=C(NO)c1cnc(NC2(c3ccc(F)cc3)CCCCC2)nc1] | 100 |
| 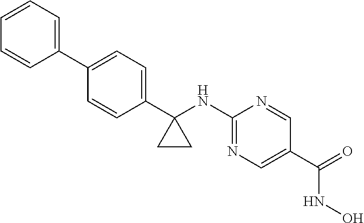 [SMILES: O=C(NO)c1cnc(NC2(c3ccc(-c4ccccc4)cc3)CC2)nc1] | 117 |

In certain embodiments, the compound of the invention is selected from Table 2, or pharmaceutically acceptable salts, esters or prodrugs thereof:

TABLE 2

| | |
|---|---|
| 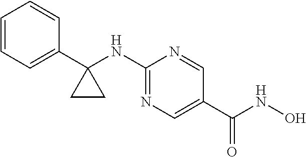 [SMILES: O=C(NO)c1cnc(NC2(c3ccccc3)CC2)nc1] | 73 |
| (ACY-738) | |
| 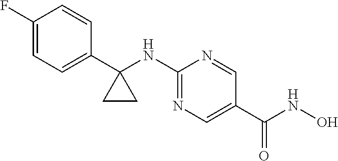 [SMILES: O=C(NO)c1cnc(NC2(c3ccc(F)cc3)CC2)nc1] | 81 |
| 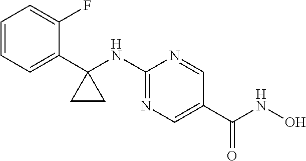 [SMILES: O=C(NO)c1cnc(NC2(c3ccccc3F)CC2)nc1] | 82 |
| 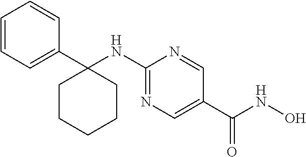 [SMILES: O=C(NO)c1cnc(NC2(c3ccccc3)CCCCC2)nc1] | 84 |
| 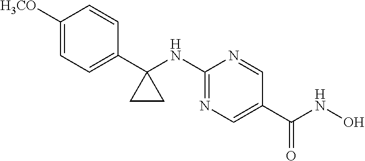 [SMILES: COc1ccc(C2(Nc3ncc(C(=O)NO)cn3)CC2)cc1] | 97 |
| 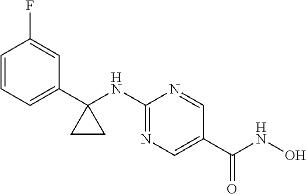 [SMILES: O=C(NO)c1cnc(NC2(c3cccc(F)c3)CC2)nc1] | 87 |
| 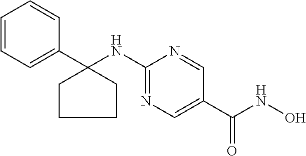 [SMILES: O=C(NO)c1cnc(NC2(c3ccccc3)CCCC2)nc1] | 88 |
| 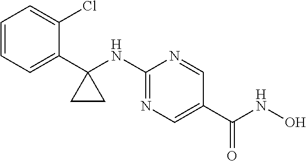 [SMILES: O=C(NO)c1cnc(NC2(c3ccccc3Cl)CC2)nc1] | 90 |
| 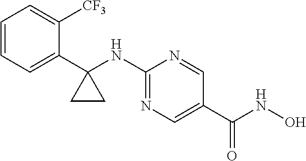 [SMILES: O=C(NO)c1cnc(NC2(c3ccccc3C(F)(F)F)CC2)nc1] | 91 |
| 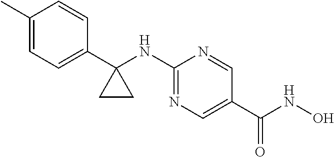 [SMILES: Cc1ccc(C2(Nc3ncc(C(=O)NO)cn3)CC2)cc1] | 93 |
| 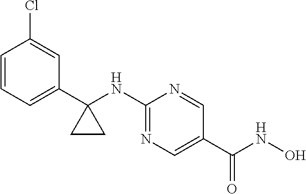 [SMILES: O=C(NO)c1cnc(NC2(c3cccc(Cl)c3)CC2)nc1] | 94 |

In a particular embodiment, the compound of Formula I is the compound 73, or a pharmaceutically acceptable salt, ester or prodrug thereof:

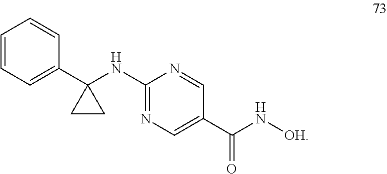 [SMILES: O=C(NO)c1cnc(NC2(c3ccccc3)CC2)nc1]

In another particular embodiment, the compound of Formula I is the compound 101, or a pharmaceutically acceptable salt, ester or prodrug thereof:

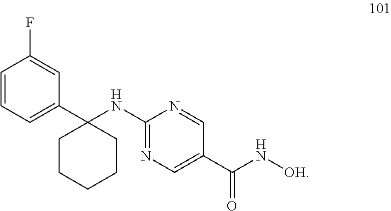 [SMILES: O=C(NO)c1cnc(NC2(c3cccc(F)c3)CCCCC2)nc1]

In another aspect, the invention provides a pharmaceutical composition comprising a compound of formula I:

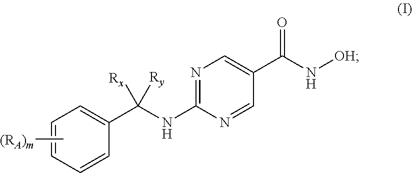

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

In certain embodiments, provided herein are methods for treating diseases or conditions mediated by HDAC6 in a subject in need thereof, comprising administering to the subject a pharmaceutical composition comprising a compound of formula I, or a pharmaceutically acceptable salt, ester or prodrug thereof.

In other embodiments, provided herein are methods for treating CNS diseases or conditions mediated by HDAC6 in a subject in need thereof, comprising administering to the subject a pharmaceutical compositions comprising a compound of formula I, or a pharmaceutically acceptable salt, ester or prodrug thereof.

In other embodiments, provided herein are methods for increasing α-tubulin acetylation in the brain of a subject in need thereof, comprising administering to the subject a pharmaceutical composition comprising a compound of formula I, or a pharmaceutically acceptable salt, ester or prodrug thereof.

In certain particular embodiments, provided herein are methods for treating depressions in a subject in need thereof, comprising administering to the subject a pharmaceutical composition comprising a compound of formula I, or a pharmaceutically acceptable salt, ester or prodrug thereof.

In other particular embodiments, provided herein are methods for increasing anxiety in a subject in need thereof, comprising administering to the subject a pharmaceutical composition comprising a compound of formula I, or a pharmaceutically acceptable salt, ester or prodrug thereof.

In preferred embodiments, a compound useful in the invention has one or more of the following properties: the compound is capable of inhibiting at least one histone deacetylase; the compound is capable of inhibiting HDAC6; the compound is a selective HDAC6 inhibitor; the compound is able to penetrate the blood-brain barrier; and/or the compound binds to the poly-ubiquitin binding domain of HDAC6.

In certain preferred embodiments, a compound of the invention comprises a metal binding moiety, preferably a zinc-binding moiety such as a hydroxamate. As noted above, certain hydroxamates are potent inhibitors of HDAC6 activity; without wishing to be bound by theory, it is believed that the potency of these hydroxamates is due, at least in part, to the ability of the compounds to bind zinc. In preferred embodiments, a compound of the invention includes at least one portion or region which can confer selectivity for a biological target implicated in a biological pathway, e.g., a biological target having tubulin deacetylase (TDAC) or HDAC activity, e.g., HDAC6. Thus, in certain preferred embodiments, a compound of the invention includes a zinc-binding moiety spaced from other portions of the molecule which are responsible for binding to the biological target.

The invention also provides for a pharmaceutical composition comprising a compound of formula I, or a pharmaceutically acceptable ester, salt, or prodrug thereof, together with a pharmaceutically acceptable carrier.

Another object of the present invention is the use of a compound as described herein in the manufacture of a medicament for use in the treatment of a disorder or disease herein. Another object of the present invention is the use of a compound as described herein for use in the treatment of a disorder or disease herein.

The synthesis of the compounds of the invention can be found in the Examples below.

Another embodiment is a method of making a compound of formula I using any one, or combination of, reactions delineated herein. The method can include the use of one or more intermediates or chemical reagents delineated herein.

Another aspect is an isotopically labeled compound of formula I delineated herein. Such compounds have one or more isotope atoms which may or may not be radioactive (e.g., ³H, ²H, ¹⁴C, ¹³C, ³⁵S, ³²P, ¹²⁵I and ¹³¹I) introduced into the compound. Such compounds are useful for drug metabolism studies and diagnostics, as well as therapeutic applications.

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e g, ammonium hydroxide solution, sodium hydroxide, and the like). A compound of the invention in a base addition salt form can be converted to the corresponding free acid by treating with a suitable acid (e.g., hydrochloric acid, etc.).

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999, and subsequent editions thereof.

Compounds of the present invention can be conveniently prepared or formed during the process of the invention, as solvates (e.g., hydrates). Hydrates of compounds of the present invention can be conveniently prepared by recrystallization from an aqueous/organic solvent mixture, using organic solvents such as dioxan, tetrahydrofuran or methanol.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included in the present invention. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds of this invention may also be represented in multiple tautomeric forms, in such instances, the invention expressly includes all tautomeric forms of the compounds described herein. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present invention. All crystal forms of the compounds described herein are expressly included in the present invention.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In embodiments, the invention provides for the intermediate compounds of the formulae delineated herein and methods of converting such compounds to compounds of the formulae herein (e.g., in schemes herein) comprising reacting a compound herein with one or more reagents in one or more chemical transformations (including those provided herein) to thereby provide the compound of any of the formulae herein or an intermediate compound thereof.

The synthetic methods described herein may also additionally include steps, either before or after any of the steps described in any scheme, to add or remove suitable protecting groups in order to ultimately allow synthesis of the compound of the formulae described herein. The methods delineated herein contemplate converting compounds of one formula to compounds of another formula (e.g., in Scheme A, A1 to A2; A2 to A3; A1 to A3). The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, sublimation, crystallization, trituration, solid phase extraction, and chromatography).

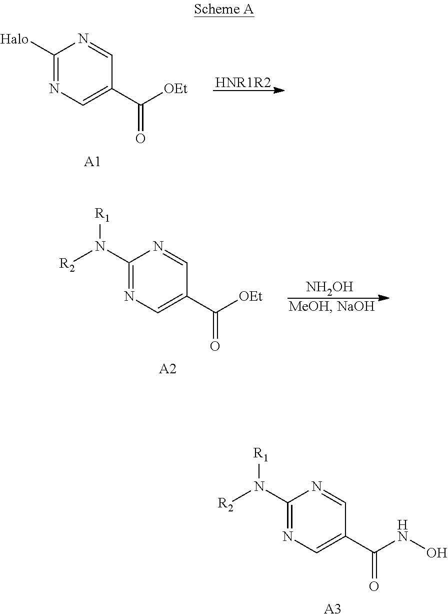

The compounds of this invention may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof. The recitation of an embodiment for a variable herein includes that embodiment as any single embodiment or in combination with any other embodiments or portions thereof.

Methods of the Invention

In another aspect, the invention provides a method of selectively inhibiting HDAC6 over other HDACs (e.g. HDAC1, HDAC2, HDAC3) in a subject, comprising administering to the subject a compound of formula I:

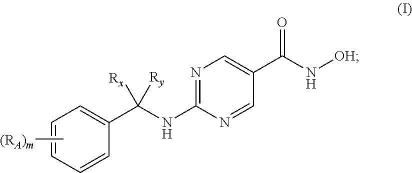

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

In one embodiment, the compound of formula I has a selectivity for HDAC6 of 5 to 1000 fold over other HDACs.

In another embodiment, the compound of formula I has a selectivity for HDAC6 when tested in a HDAC enzyme assay of about 5 to 1000 fold over other HDACs.

In certain embodiments, the compound has a selectivity for HDAC6 of 15 to 40 fold over other HDACs.

In another aspect, the invention provides a method of treating depression and/or anxiety in a subject comprising administering to the subject a compound of formula I:

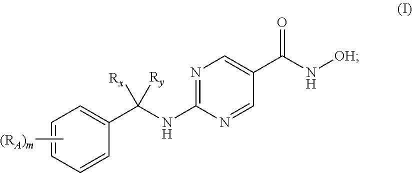

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

In certain embodiments, the depression is one or more of the following: major depression, clinical depression, chronic depression, dysthymia, atypical depression, bipolar depression, manic depression, seasonal depression, phychotic depression, and postpartum depression.

In other embodiments, the anxiety is one or more of the following: generalized anxiety disorder, obsessive-compulsive disorder (OCD), panic disorder, post-traumatic stress disorder (PTSD), and social phobia (or social anxiety disorder).

In another aspect, the invention provides a method of treating a disease mediated by HDAC6 in a subject comprising administering to the subject a compound of formula I:

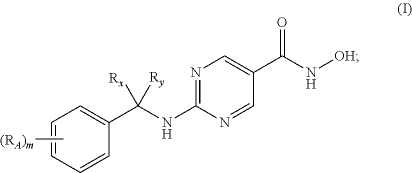

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2.

In one embodiment, Rₓ and R_{y} together with the carbon to which each is attached, forms a cyclopropyl, cyclopentyl, cyclohexyl, or tetrahydropyran.

In another embodiment, Rₓ and R_{y} together with the carbon to which each is attached, forms a cyclopropyl or cyclohexyl.

In another embodiment, m is 1 or 2, and each R_{A} is independently methyl, phenyl, F, Cl, methoxyl, or CF₃.

In other embodiments, m is 0.

In certain embodiments, the disease mediated by HDAC6 is depression, major depression, clinical depression, chronic depression, dysthymia, atypical depression, bipolar depression, manic depression, seasonal depression, phychotic depression, postpartum depression, anxiety, generalized anxiety disorder, obsessive-compulsive disorder (OCD), panic disorder, post-traumatic stress disorder (PTSD), and social phobia (or social anxiety disorder).

In one embodiment, the HDAC6 inhibitors of the invention are useful for treating any one or more of the following: depression, major depression, clinical depression, chronic depression, dysthymia, atypical depression, bipolar depression, manic depression, seasonal depression, phychotic depression, postpartum depression, anxiety, generalized anxiety disorder, obsessive-compulsive disorder (OCD), panic disorder, post-traumatic stress disorder (PTSD), and social phobia (or social anxiety disorder).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

Preferably, the HDAC6 inhibitors are selective inhibitors of HDAC6 and, as such, are useful in the treatment of disorders modulated by histone deacetylases. In one embodiment, the HDAC6 inhibitors of the invention are selective inhibitors of tubulin deacetylases and, as such, are useful in the treatment of disorders modulated by tubulin deacetylases.

Preferably, the HDAC6 inhibitors penetrate the blood-brain barrier.

Thus, in another aspect of the invention, methods for the treatment of depression and/or anxiety are provided comprising administering a therapeutically effective amount of an HDAC6 inhibitor, as described herein, to a subject in need thereof. In certain embodiments, the subject is identified as in need of such treatment. In certain embodiments, a method for the treatment of a diseases is provided comprising administering a therapeutically effective amount of an HDAC6 inhibitor, or a pharmaceutical composition comprising an HDAC6 inhibitor to a subject in need thereof, in such amounts and for such time as is necessary to achieve the desired result. In certain embodiments of the present invention a “therapeutically effective amount” of an HDAC6 inhibitor or pharmaceutical composition is that amount effective for modulating a subset of a group of cytokines according to the invention.

In certain embodiments, the method involves the administration of a therapeutically effective amount of an HDAC6 inhibitor or a pharmaceutically acceptable derivative thereof to a subject (including, but not limited to a human or animal) in need of it (including a subject identified as in need). In certain embodiments, the HDAC6 inhibitors are useful for the treatment of depression, including major depression, clinical depression, chronic depression, dysthymia, atypical depression, bipolar depression, manic depression, seasonal depression, phychotic depression, postpartum depression, anxiety, generalized anxiety disorder, obsessive-compulsive disorder (OCD), panic disorder, post-traumatic stress disorder (PTSD), and social phobia (or social anxiety disorder).

In certain embodiments, the invention provides a method of treatment of any of the disorders described herein, wherein the subject is a human.

In accordance with the foregoing, the present invention further provides a method for preventing or treating any of the diseases or disorders described above in a subject in need of such treatment, which method comprises administering to said subject a therapeutically effective amount of an HDAC6 inhibitor of the invention or a pharmaceutically acceptable salt thereof. For any of the above uses, the required dosage will vary depending on the mode of administration, the particular condition to be treated and the effect desired.

In another embodiment, the methods herein comprise a step wherein a compound is identified as having a Z-fold selectivity for HDAC6 over other HDACs (e.g., HDAC1, HDAC2, HDAC3), wherein Z is an integer between 2 and 1000.

In one embodiment, the invention provides a method further comprising co-administering one or more of a chemotherapeutic agent, radiation agent, hormonal agent, biological agent, or an anti-inflammatory agent to the subject.

In another aspect, the invention provides a kit comprising a compound capable of treating depression and/or anxiety in a subject selected from one or more compounds of formula I,

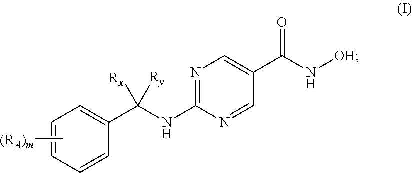

or a pharmaceutically acceptable salt, ester or prodrug thereof,

wherein,

Rₓ and R_{y} together with the carbon to which each is attached, forms a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;

each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and

m is 0, 1, or 2;

and instructions for use in treating depression and/or anxiety.

As discussed above, the present invention provides compounds useful for the treatment of depression and/or anxiety. In certain embodiments, the compounds of the present invention are useful as inhibitors of histone or tubulin deacetylases. In certain exemplary embodiments, the compounds of the invention are useful for disorders associated with tubulin deacetylation activity.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

As discussed above, the compounds of the invention are selective inhibitors of HDAC6 and, as such, are useful in the treatment of disorders modulated by histone deacetylases. As discussed above, the compounds of the invention are selective inhibitors of tubulin deacetylases and, as such, are useful in the treatment of disorders modulated by tubulin deacetylases. For example, compounds of the invention may be useful in the treatment of depression and/or anxiety.

Thus, in another aspect of the invention, methods for the treatment of depression and/or anxiety are provided comprising administering a therapeutically effective amount of a compound, as described herein, to a subject in need thereof. In certain embodiments, the subject is identified as in need of such treatment. In certain embodiments, a method for the treatment of depression and/or anxiety is provided comprising administering a therapeutically effective amount of a compound, or a pharmaceutical composition comprising a compound to a subject in need thereof, in such amounts and for such time as is necessary to achieve the desired result. In certain embodiments of the present invention a “therapeutically effective amount” of the compound or pharmaceutical composition is that amount effective for treating depression and/or anxiety. The compounds and compositions, according to the method of the present invention, may be administered using any amount and any route of administration effective for treating depression and/or anxiety. Thus, the expression “amount effective to treat depression,” as used herein, refers to a sufficient amount of agent to treat the signs or symptoms of depression. Likewise, the expression “amount effective to treat anxiety,” as used herein, refers to a sufficient amount of agent to treat the signs or symptoms of anxiety. The exact amount required will vary from subject to subject, depending on the species, age, and general condition of the subject, the severity of the infection, the particular anticancer agent, its mode of administration, and the like.

In certain embodiments, the method involves the administration of a therapeutically effective amount of the compound or a pharmaceutically acceptable derivative thereof to a subject (including, but not limited to a human or animal) in need of it.

In accordance with the foregoing, the present invention further provides a method for preventing or treating any of the diseases or disorders described above in a subject in need of such treatment, which method comprises administering to said subject a therapeutically effective amount of a compound of the invention or a pharmaceutically acceptable salt thereof. For any of the above uses, the required dosage will vary depending on the mode of administration, the particular condition to be treated and the effect desired.

Pharmaceutical Compositions

In another aspect, the invention provides a pharmaceutical composition comprising a compound of formula I, or a pharmaceutically acceptable ester, salt, or prodrug thereof, together with a pharmaceutically acceptable carrier. This pharmaceutical composition can be used in the treatment of depression and/or anxiety.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects (e.g., control of any adverse effects).

In certain embodiments, the pharmaceutical compositions of the present invention further comprise one or more additional therapeutically active ingredients. For purposes of the invention, the term “palliative” refers to treatment that is focused on the relief of symptoms of a disease and/or side effects of a therapeutic regimen, but is not curative. For example, palliative treatment encompasses painkillers, antinausea medications, anti-pyretics, and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

The pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Solid compositions of a similar type may also be employed as fillers in soft and hard-filled gelatin capsules using such excipients as lactose or milk sugar as well as high molecular weight polyethylene glycols and the like.

According to the methods of treatment of the present invention, disorders are treated or prevented in a subject, such as a human or other animal, by administering to the subject a therapeutically effective amount of a compound of the invention, in such amounts and for such time as is necessary to achieve the desired result. The term “therapeutically effective amount” of a compound of the invention, as used herein, means a sufficient amount of the compound so as to decrease the symptoms of a disorder in a subject. As is well understood in the medical arts a therapeutically effective amount of a compound of this invention will be at a reasonable benefit/risk ratio applicable to any medical treatment.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in controlled release form. Suitable unit dosage forms for oral administration comprise from ca. 1 to 50 mg active ingredient.

In certain embodiments, a therapeutic amount or dose of the compounds of the present invention may range from about 0.1 mg/kg to about 500 mg/kg (about 0.18 mg/m² to about 900 mg/m²), alternatively from about 1 to about 50 mg/kg (about 1.8 to about 90 mg/m²). In general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### EXAMPLES

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. Definitions of variables in the structures in schemes herein are commensurate with those of corresponding positions in the formulae delineated herein.

The synthesis of the compounds of the invention is provided in PCT/US2011/060791, which is incorporated herein by reference in its entirety.

### Example 1

### Synthesis of Compound 1

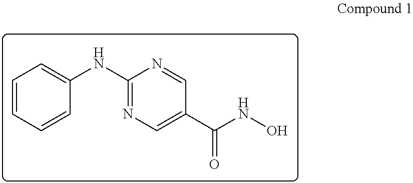 [SMILES: O=C(NO)c1cnc(Nc2ccccc2)nc1]

### Synthetic Scheme:

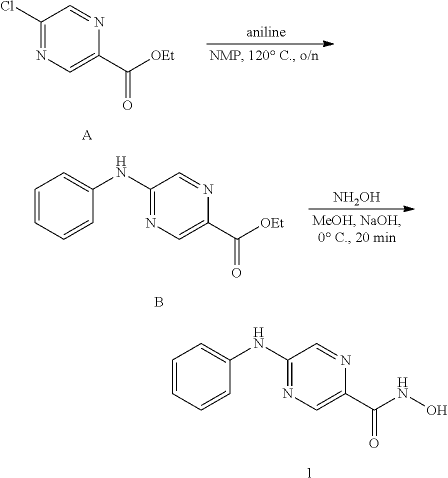

### Synthesis of ethyl 2-(phenylamino)pyrimidine-5-carboxylate (Compound B)

To a solution of compound A in NMP (20 ml) was added aniline (1.0 g, 5.36 mmol) and heated at 120° C. for overnight. After the reaction completed, the mixture was poured into ice water (20 ml), the precipitate was collected and washed with methanol (5 ml*2), the precipitation was isolated by filtration to afford the compound B as a pale solid (980 mg, 75%).

### Synthesis of N-hydroxy-2-(phenylamino)pyrimidine-5-carboxamide (Compound 1)

To a solution of compound B (100 mg, 0.411 mmol) in methanol/dichloromethane (5/2 ml) was added 98% NH₂OH (1.7 ml, 50% in water) at 0° C. and stirred for 10 min. A solution of saturated sodium hydroxide in methanol (1.5 ml) was added and the mixture kept stirring for another 10 min. The reaction mixture was concentrated in vacuum and the crude residue was acidified to pH=6-7 with 2N HCl. The precipitate was collected, washed with water to afford 1 as an off-white solid (90 mg, 94.7%).

### Example 2

### Synthesis of Compound 99

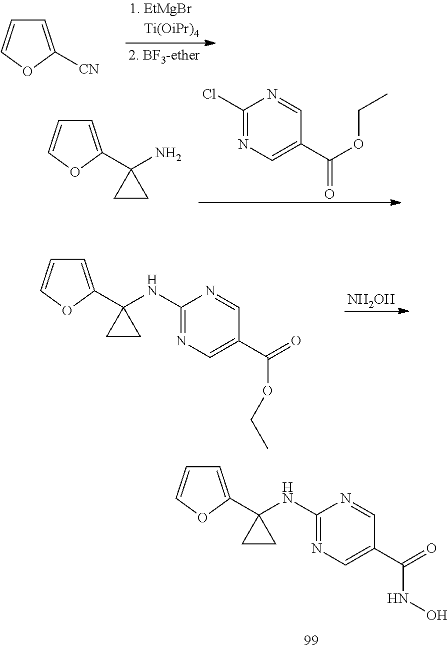 [SMILES: N#Cc1ccco1 ; NC1(c2ccco2)CC1 ; CCOC(=O)c1cnc(Cl)nc1 ; CCOC(=O)c1cnc(NC2(c3ccco3)CC2)nc1 ; "NH2OH" ; O=C(NO)c1cnc(NC2(c3ccco3)CC2)nc1]

### Example 3

### Synthesis of Compound 110

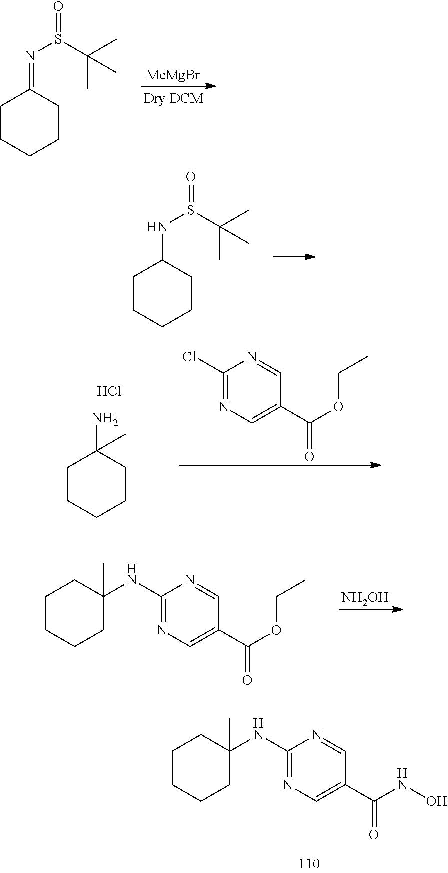

### Example 4

### Synthesis of Compound 111

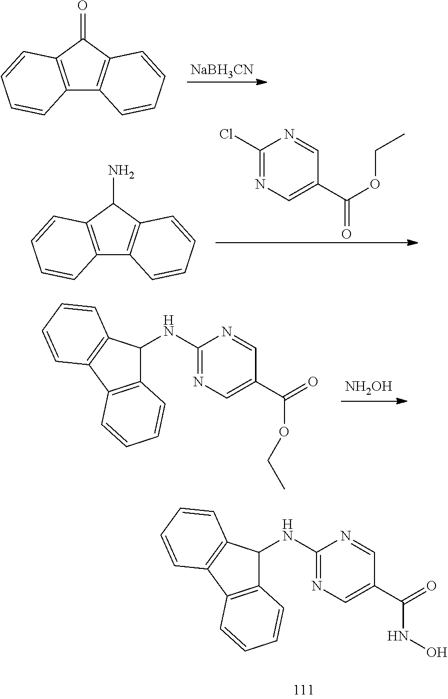

### Example 5

### Synthesis of 2-((1-(3-fluorophenyl)cyclohexyl)amino)-N-hydroxypyrimidine-5-carboxamide (Compound 101/ACY-775)

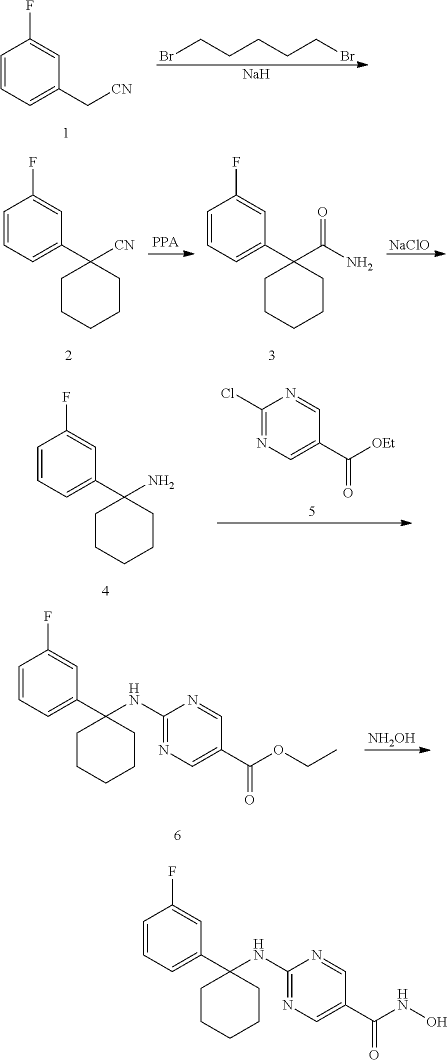

### Synthesis of Intermediate 2

To a solution of compound 1 (100 g, 0.74 mol) in dry DMF (1000 ml) was added 1,5-dibromopentane (170 g, 0.74 mol). NaH (65 g, 2.2 eq) was added dropwise while the reaction was cooled in an ice bath. The resulting mixture was vigorously stirred overnight at 50° C. The suspension was carefully quenched with ice water and extracted with ethyl acetate (3×500 ml). The combined organic layers were concentrated to afford the crude product, which was purified by flash column chromatography to give compound 2 as pale solid (100 g, 67%).

### Synthesis of Intermediate 3

A solution of compound 2 (100 g, 0.49 mol) in PPA (500 ml) was heated at 110° C. for about 5-6 hours. After completion, the resulting mixture was carefully adjusted to a pH of about 8-9 with sat.NaHCO₃ solution. The resulting precipitate was collected and washed with water (1000 ml) to afford compound 3 as white solid (95 g, 87%).

### Synthesis of Intermediate 4

To a solution of compound 3 (95 g, 0.43 mol) in n-BuOH (800 ml) was added NaClO (260 ml, 1.4 eq). 3N NaOH (400 ml, 2.8 equiv.) was then added at 0° C. and the reaction was stirred overnight at r.t. The resulting mixture was extracted with EA (2×500 ml), and the combined organic layers washed with brine. The solvent was removed in vacuo to afford the crude product which was further purified by treatment with HCl salt to yield compound 4 as a white powder (72 g, 73%).

### Synthesis of Intermediate 6

To a solution of compound 4 (2.29 g 10 mmol) in dioxane (50 ml) was added compound 5 (1.87 g, 1.0 equiv.) and DIPEA (2.58 g, 2.0 equiv.). The mixture was heated overnight at 110-120° C. The resulting mixture was directly purified on silica gel column to afford the coupled product, compound 6, as a white solid (1.37 g, 40%).

### Synthesis of 2-((1-(3-fluorophenyl)cyclohexyl)amino)-N-hydroxypyrimidine-5-carboxamide (Compound 101/ACY-775)

To a solution of compound 6 (100 mg, 0.29 mmol) in MeOH/DCM (10 ml, 1:1) was added 50% NH₂OH in water (2 ml, excess). Sat. NaOH in MeOH (2 ml, excess) was then added at 0° C. and the reaction was stirred for 3-4 hours. After completion, the resulting mixture was concentrated and acidified with 2N HCl to reach a pH of 4-5. The precipitate was collected and washed with water (10 ml) to remove excess NH₂OH. Drying the precipitate afforded 2-((1-(3-fluorophenyl)cyclohexyl)amino)-N-hydroxypyrimidine-5-carboxamide as a white powder (70 mg, 73%).

### Example 6

### Synthesis of N-hydroxy-2-((1-phenylcyclopropyl)amino)pyrimidine-5-carboxamide (Compound 73/ACY-738)

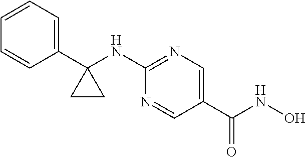 [SMILES: O=C(NO)c1cnc(NC2(c3ccccc3)CC2)nc1]

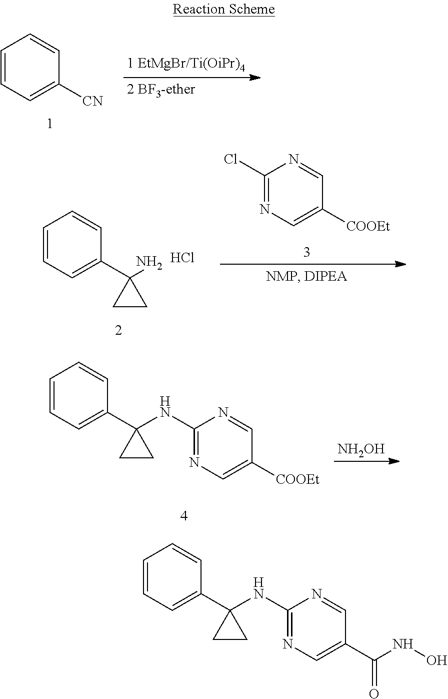

### Synthesis of Intermediate 2

A solution of compound 1, benzonitrile, (250 g, 1.0 equiv.), and Ti(OiPr)₄ (1330 ml, 1.5 equiv.) in MBTE (3750 ml) was cooled to about −10 to −5° C. under a nitrogen atmosphere. EtMgBr (1610 ml, 3.0M, 2.3 equiv.) was added dropwise over a period of 60 min, during which the inner temperature of the reaction was kept below 5° C. The reaction mixture was allowed to warm to 15-20° C. for 1 hr. BF₃-ether (1300 ml, 2.0 equiv.) was added dropwise over a period of 60 min, while the inner temperature was maintained below 15° C. The reaction mixture was stirred at 15-20° C. for 1-2 hr. and stopped when a low level of benzonitrile remained. 1N HCl (2500 ml) was added dropwise while maintaining the inner temperature below 30° C. NaOH (20%, 3000 ml) was added dropwise to bring the pH to about 9.0, while still maintaining a temperature below 30° C. The reaction mixture was extracted with MTBE (3 L×2) and EtOAc (3 L×2), and the combined organic layers were dried with anhydrous Na₂SO₄ and concentrated under reduced pressure (below 45° C.) to yield a red oil. MTBE (2500 ml) was added to the oil to give a clear solution, and upon bubbling with dry HCl gas, a solid precipitated. This solid was filtered and dried in vacuum yielding 143 g of compound 2.

### Synthesis of Intermediate 4

Compound 2 (620 g, 1.0 equiv) and DIPEA (1080 g, 2.2 equiv. were dissolved in NMP (3100 ml) and stirred for 20 min. Compound 3 (680 g, 1.02 equiv.) was added and the reaction mixture was heated to about 85-95° C. for 4 hrs. The solution was allowed to slowly cool to r.t. This solution was poured onto H₂O (20 L) and much of the solid was precipitated out from the solution with strong stirring. The mixture was filtered and the cake was dried under reduced pressure at 50° C. for 24 hr., yielding 896 g of compound 4 (solid, 86.8%).

### Synthesis of N-hydroxy-2-((1-phenylcyclopropyl)amino)pyrimidine-5-carboxamide (Compound 73/ACY-738)

A solution of MeOH (1000 ml) was cooled to about 0-5° C. with stirring. NH₂OH HCl (1107 g, 10 equiv.) was added, followed by careful addition of NaOCH₃ (1000 g, 12.0 equiv.) The resulting mixture was stirred at 0-5° C. for one hr, and was filtered to remove the solid. Compound 4 (450 g, 1.0 equiv.) was added to the reaction mixture in one portion, and stirred at 10° C. for two hours until compound 4 was consumed. The reaction mixture was adjusted to a pH of about 8.5-9 through addition of HCl (6N), resulting in precipitation. The mixture was concentrated under reduced pressure. Water (3000 ml) was added to the residue with intense stirring and the precipitate was collected by filtration. The product was dried in an oven at 45° C. overnight (340 g, 79% yield).

### Example 7

### HDAC Enzyme Assays

Compounds for testing were diluted in DMSO to 50 fold the final concentration and a ten point three fold dilution series was made. The compounds were diluted in assay buffer (50 mM HEPES, pH 7.4, 100 mM KCl, 0.001% Tween-20, 0.05% BSA, 20 μM TCEP) to 6 fold their final concentration. The HDAC enzymes (purchased from BPS Biosciences) were diluted to 1.5 fold their final concentration in assay buffer. The tripeptide substrate and trypsin at 0.05 μM final concentration were diluted in assay buffer at 6 fold their final concentration. The final enzyme concentrations used in these assays were 3.3 ng/ml (HDAC1), 0.2 ng/ml (HDAC2), 0.08 ng/ml (HDAC3) and 2 ng/ml (HDAC6). The final substrate concentrations used were 16 μM (HDAC1), 10 μM (HDAC2), 17 μM (HDAC3) and 14 μM (HDAC6). Five μl of compounds and 20 μl of enzyme were added to wells of a black, opaque 384 well plate in duplicate. Enzyme and compound were incubated together at room temperature for 10 minutes. Five μl of substrate was added to each well, the plate was shaken for 60 seconds and placed into a Victor 2 microtiter plate reader. The development of fluorescence was monitored for 60 min and the linear rate of the reaction was calculated. The IC₅₀ was determined using Graph Pad Prism by a four parameter curve fit.

| | IC50 (nm) | | | | | | |
|---|---|---|---|---|---|---|---|
| Compound ID | HDAC1 | | HDAC2 | | HDAC3 | | HDAC6 |
| 73 (ACY-738) | 94 | (60x) | 128 | (81.9x) | 219 | (139.5x) | 2 |
| 81 | 47 | (18.3x) | 78 | (30.2x) | 120 | (46.4x) | 3 |
| 82 | 16 | (9.9x) | 18 | (11.2x) | 22 | (13.6x) | 2 |
| 84 | 2226 | (302.2x) | 2971 | (403.4x) | 4515 | (613x) | 7 |
| 87 | 85 | (45.4x) | 116 | (61.6x) | 233 | (124.1x) | 2 |
| 88 | 711 | (215.2x) | 978 | (296.1x) | 2243 | (679.1x) | 3 |
| 90 | 27 | (19.9x) | 36 | (26.2x) | 41 | (30.2x) | 1 |
| 91 | 160 | (72.3x) | 288 | (130.2x) | 374 | (169.4x) | 2 |
| 93 | 80 | (25.9x) | 107 | (34.7x) | 157 | (51x) | 3 |
| 94 | 22 | (12x) | 32 | (17.4x) | 50 | (27.4x) | 2 |
| 97 | 19 | (8.3x) | 28 | (12.1x) | 37 | (16.4x) | 2 |
| 100 | 3206 | (339.9x) | 4761 | (504.7x) | 13474 | (1428.4x) | 9 |
| 101 (ACY-775) | 2123 | (283.5x) | 2570 | (343.2x) | 11223 | (1498.8x) | 7 |

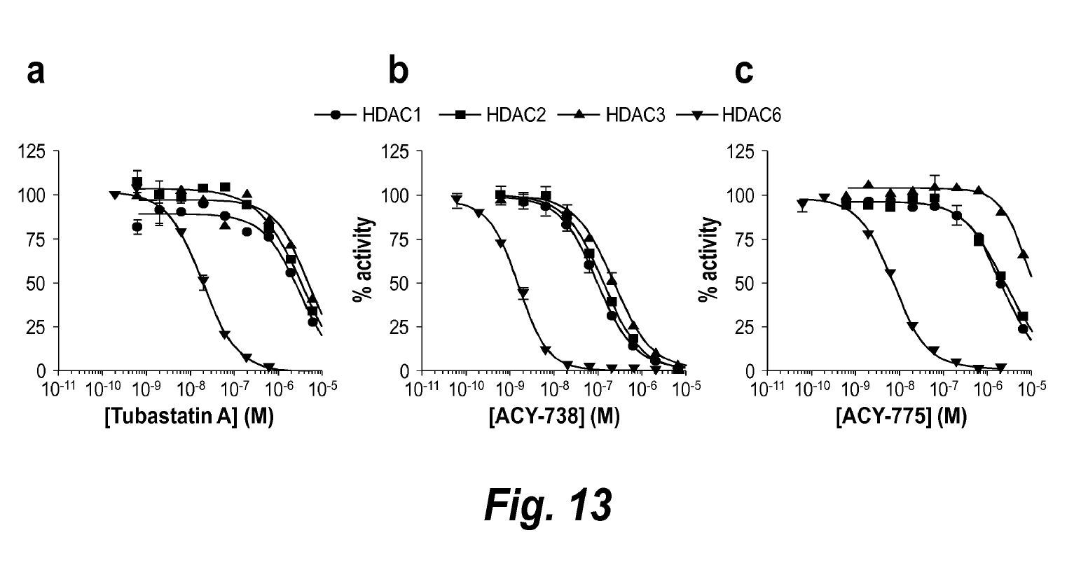

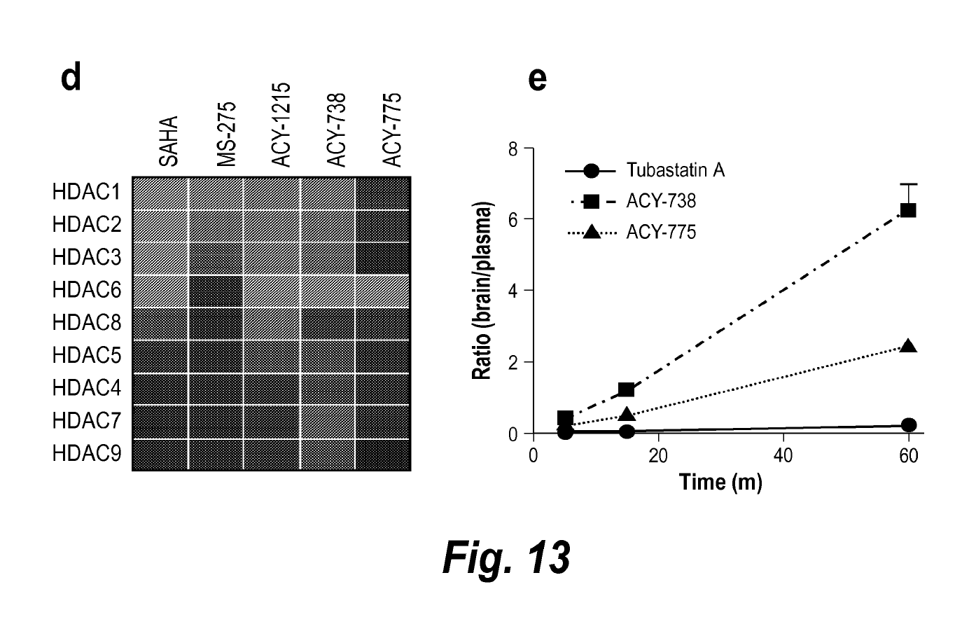

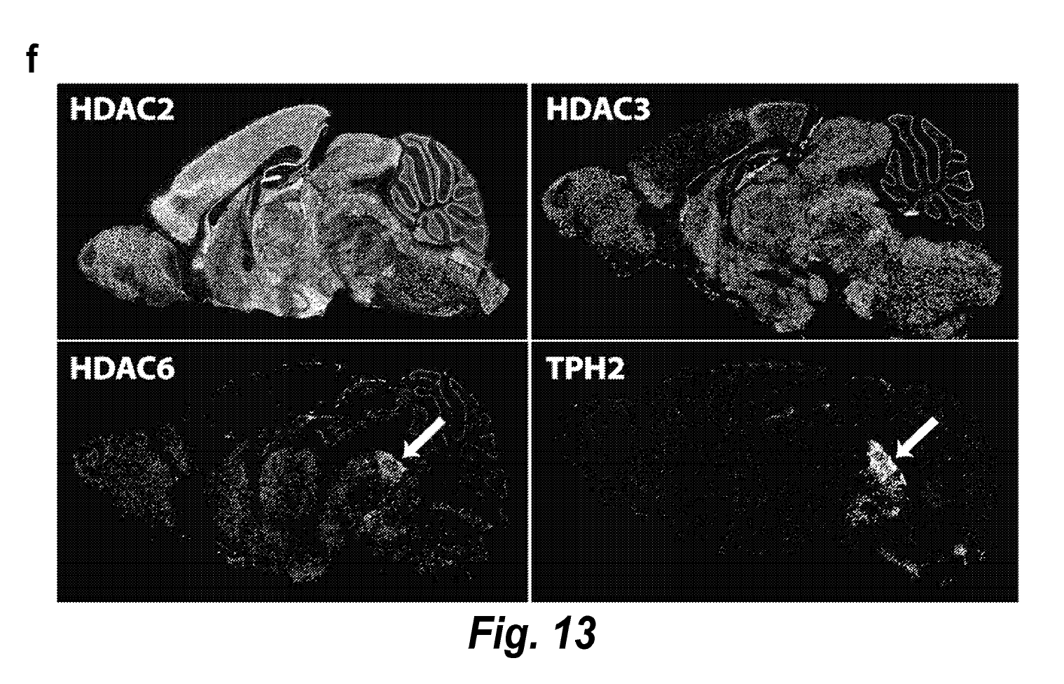

Data in FIGS. 13a-13d also show selectivity, potency and pharmacokinetic properties of selected HDAC6 inhibitors. FIG. 13a shows the dose-dependent enzymatic inhibition of recombinant HDAC1, HDAC2, HDAC3 and HDAC6 incubated with tubastatin A. FIG. 13b shows the dose-dependent enzymatic inhibition of recombinant HDAC1, HDAC2, HDAC3 and HDAC6 incubated with compound 73. FIG. 13c shows the dose-dependent enzymatic inhibition of recombinant HDAC1, HDAC2, HDAC3 and HDAC6 incubated with compound 101. FIG. 13d shows the heat map summary of 1050 values for the inhibitors compound 73 and compound 101 and reference compounds SAHA, MS-275 and ACY-1215 (n=2 per condition): the brighter reds denote higher inhibitory potency of the compounds on the corresponding HDAC isoform, and the black background indicates lack of activity. FIG. 13e shows the ratios of brain concentration to plasma concentration over time after acute 5 mg/kg i.p. injection.

### Conclusions: Inhibition is Selective for HDAC6 and Superior to Tubastatin A

Pan-I-I:DAC inhibitors lead to major changes in gene transcription, which can have numerous undesired effects, thus making them unlikely candidates for antidepressant medications. As HDAC3 is hypothesized to be the main cause of these effects, it is of interest to separate this inhibition from that of HDAC6. The compounds tested in this study have been designed to do so, having at least 100× selectivity for HDAC6 over HDAC3. The compounds were tested for HDAC inhibition by fluorimetric assay using tripeptide substrate 3 at amounts equal to the Km of each enzyme. HDAC enzymes were pre-incubated with dilutions of each compound for 10 minutes. Fluorescence was monitored for 30 minutes after addition of substrate to the enzyme/compound mixtures and IC50 values were determined by a four-parameter curve fit.

As shown in Table 3 below, compounds 73 and 101 demonstrate inhibitory activity against recombinant HDAC6 with IC50 values of 1.7 nM and 7.5 nM, respectively, with respective average selectivity over class I HDACs being 100 and 700-fold (Table 1, FIGS. 13b, 13c, and 13d). For comparison, the reference HDAC6 inhibitor tubastatin A showed an IC50 for HDAC6 of 18 nM and displayed an average of about 200 fold selectivity over Class I HDACs (FIG. 13a). Compound 101 had minimal activity against all other class II HDAC isoforms (IC50>1 μM). The activity of compounds 73 and 101 in in vitro assays was fully explained by inhibition of the C terminal catalytic domain of HDAC6, as deacetylase activity of a point-mutant with inactive N-terminal domain (H216A) but intact C-terminal domain was comparable to HDAC6 WT and fully inhibited by compound 73. In contrast, the HDAC6 C-terminal catalytic mutant was almost completely devoid of deacetylase activity.

TABLE 3

| | HDAC1 | HDAC2 | | HDAC6 |
|---|---|---|---|---|
| | IC50 | IC50 | HDAC3 IC50 | IC50 |
| Compound | (nM) | (nM) | (nM) | (nM) |
| Tubastatin A | 3259 (172x) | 3575 (189x) | 4948 (261x) | 18.9 |
| Compound 73 | 94 (55x) | 128 (75x) | 218 (128x) | 1.7 |
| (ACY-738) | | | | |
| Compound 101 | 2123 (283x) | 2570 (342x) | 11223 (1496x) | 7.5 |
| (ACY-775) | | | | |

### Example 8

### Reversible Acetylation of α-Tubulin in Murine Models of Depression and Antidepressant Action

Various antidepressants and mood stabilizers promote protein hyperacetylation in the brain via the inhibition of histone deacetylases (HDACs), an observation suggesting HDACs may contribute to the therapeutic activity of these medications. This hypothesis is in line with the well-replicated finding that HDAC inhibitors promote antidepressant-like responses in models of depression. Although most studies in the brain have been focused on the role of this enzyme family in chromatin regulation, recent reports point to cytoskeletal proteins as another important class of HDAC substrates. α-Tubulin, a protein that becomes heavily hyperacetylated after treatment with pan-HDAC inhibitors, is a key component of microtubules with roles in activity-dependant synapse remodeling and intracellular trafficking. HDAC6, a cytoplasmic class JIB HDAC, has been identified as the primary α-tubulin deacetylase and is inhibited by certain tricyclic and SSRIs antidepressants. Additionally, it has been shown that HDAC6 knockout mice have an antidepressant-like phenotype. The aim here is to test the causal involvement of α-tubulin acetylation in the antidepressant activity of HDAC inhibitors and the pro-resilient effect of HDAC6 knockout. The changes in global levels, regional distribution and subcellular localization of α-tubulin acetylation in the brain were examined following exposure to depressogenic manipulations (ie chronic social defeat) and antidepressant treatment and show a long lasting increase in α-tubulin acetylation following chronic stress. The ability of novel pharmacological inhibitors, with enhanced HDAC6 selectivity and brain bioavailability, to produce region selective changes in α-tubulin acetylation and produce antidepressant-like responses in WT and HDAC6 KO mice was tested. Finally viral mediated approaches were used to test whether rescue of tubulin acetylation in HDAC6 KO mice can restore stress vulnerability. These studies provide critical new information about the role of α-tubulin acetylation in depression-like behaviors.

### BACKGROUND

HDAC6 is highly colocalized with Tph, a marker for serotonin cells, which are a critical target of antidepressants. HDAC6 is highly localized to the dorsal and median raphe, areas which account for the majority of ascending serotonin neurons in the brain. HDAC6 is responsible for deacetylation of alpha-tubulin, as evidenced by the increase in tubulin acetylation in fibroblasts treated with TSA, but not with trapoxin or butyrate. Additionally, siRNA knockdown of HDAC6 in fibroblasts also leads to increased acetylation. Cre driven knockdown of HDAC6 in neurons in vivo also leads to increased tubulin acetylation in mouse brain lysate. HDAC6 shows 30% decreased mRNA expression in serotonergic neurons in the raphe nucleus in mice that are spontaneously resistant to developing depressive-like behavior following chronic social defeat as well as in mice resilient after treatment with imipramine, a common tricyclic antidepressant. Serotonin cell specific knockout of HDAC6 leads to an antidepressant-like phenotype in the tail suspension test, forced swim test and social interaction test following chronic social defeat.

### Effects on Tubulin Acetylation

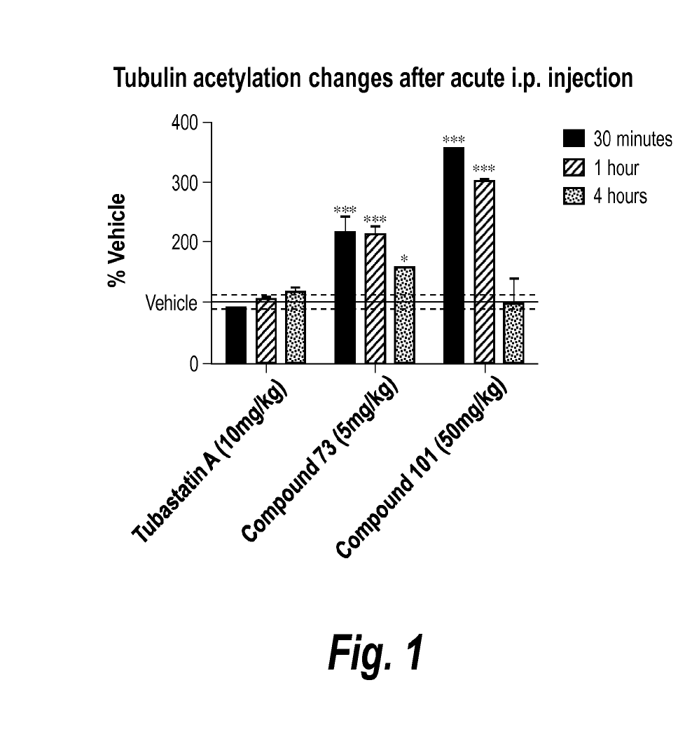

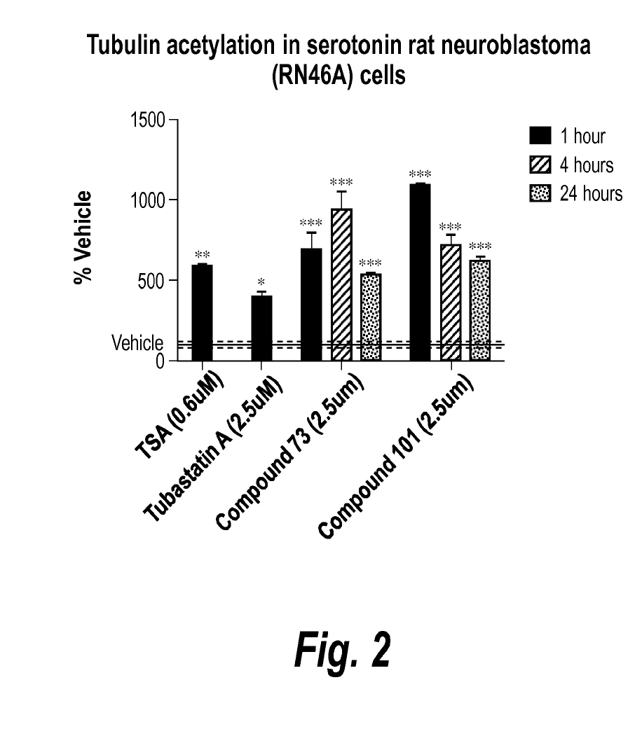

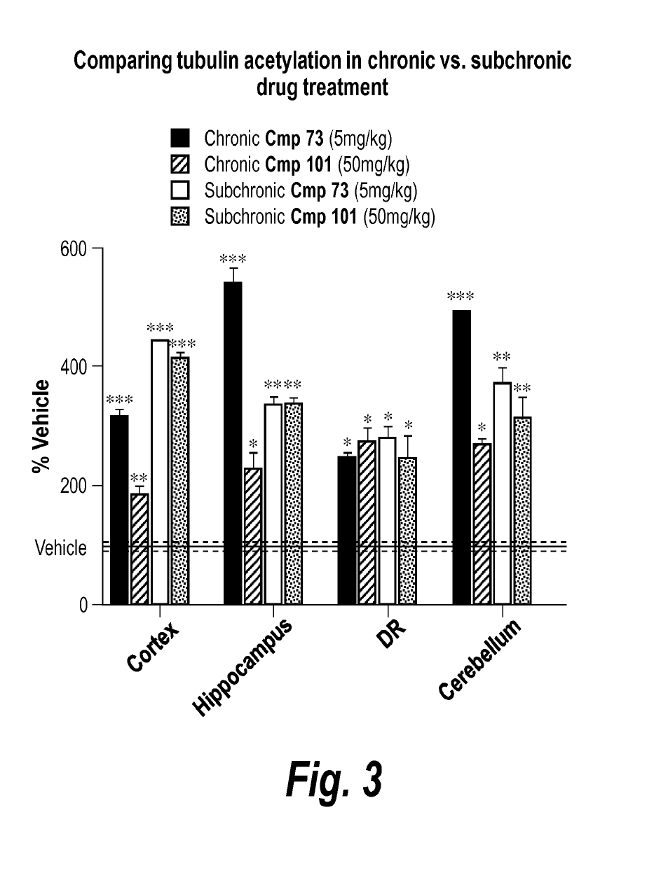

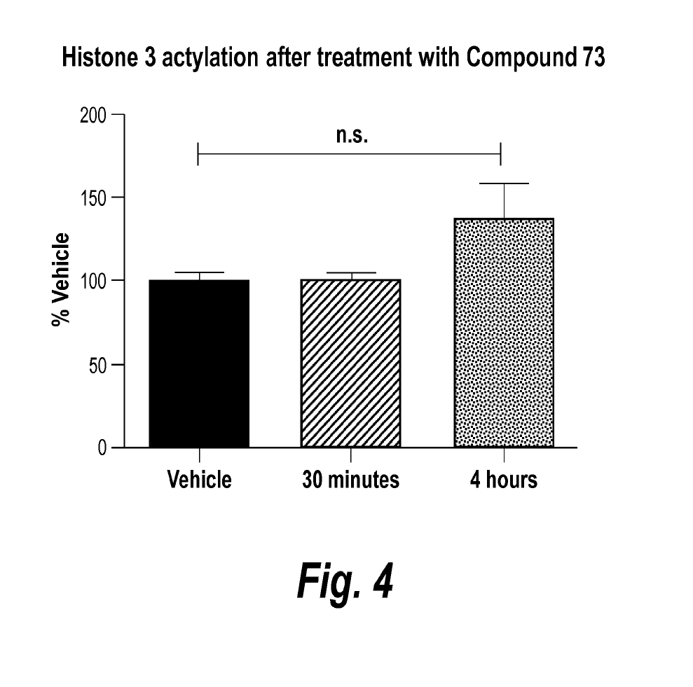

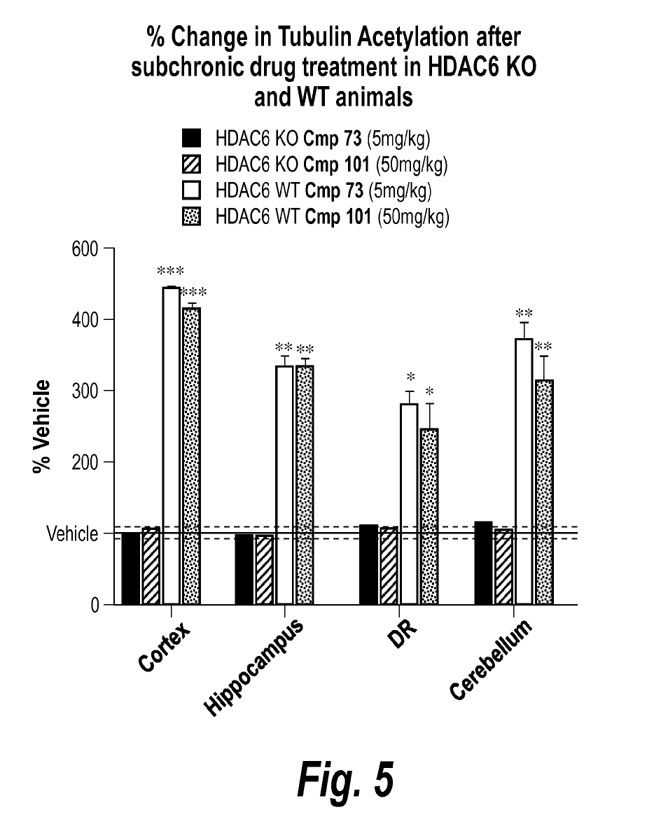

Compounds 73 and 101 led to increases in tubulin acetylation in neuronal cell culture (See FIG. 2) as well as in mouse whole brains after acute i.p. injection (See FIG. 1) and specific brain regions after repeated i.p injection (See FIGS. 5 and 3), without leading to increased histone acetylation (See FIG. 4). FIG. 5 shows that mice with neuronal HDAC6 knockout did not show a further increase in tubulin acetylation when treated with HDAC6 inhibitors, however WT mice showed large increases compared to vehicle, confirming that these increases are due to HDAC6-specific inhibition. In FIG. 3, it was observed that subchronic treatment, consisting of 5 treatments at 48, 28, 24, 4 and ½ hours before sacrifice, led to greater increase in tubulin acetylation than did chronic treatment of 1 injection/day for 21 days. For cell culture, RN46B cells were treated for with 0.25, 2.5 or 25 μM drug in DMSO for 1, 4 or 24 hours as indicated. Tukey post-hoc analysis: * P=0.01 to 0.05, ** P=0.001-0.01, ***P<0.001 when compared with vehicle treatment.

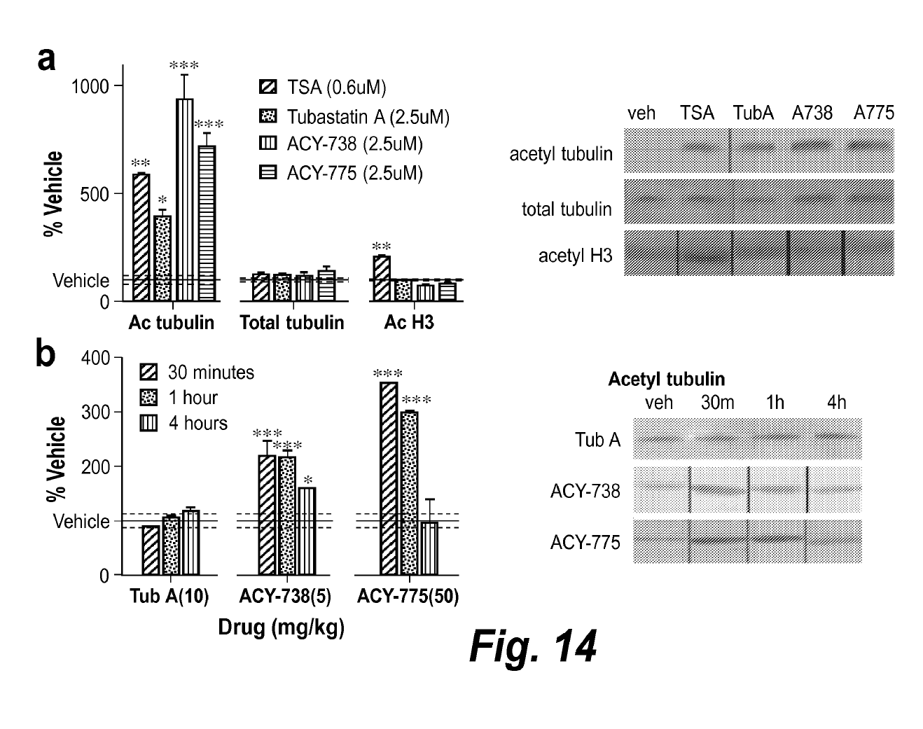

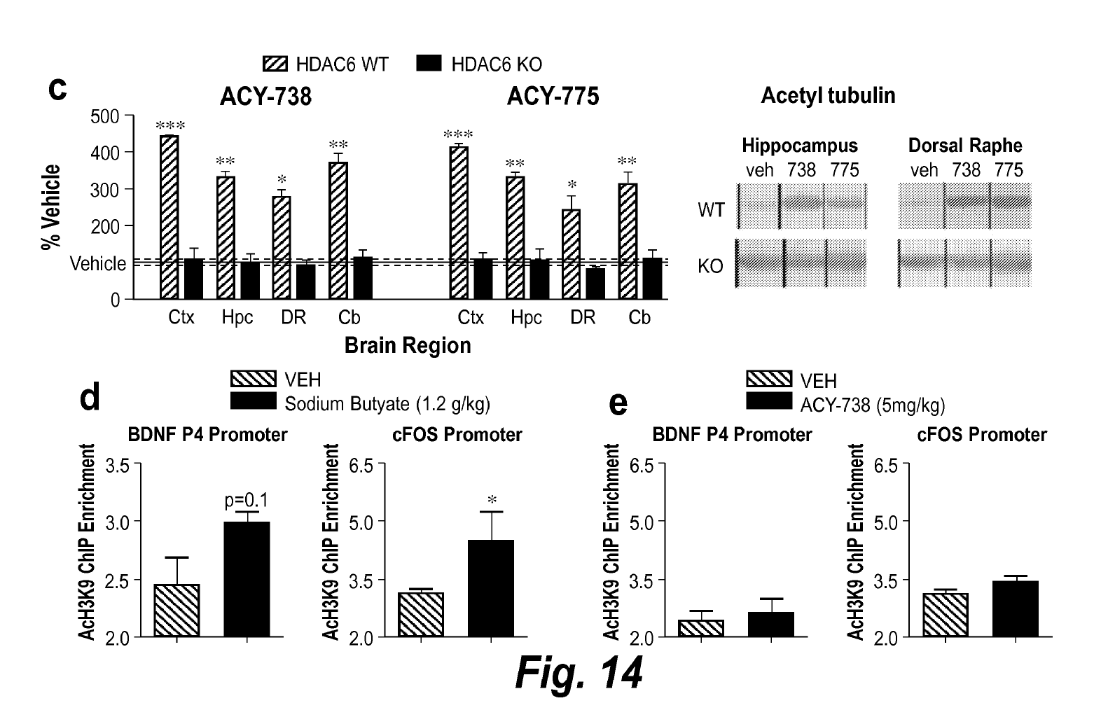

Data in FIGS. 13f and 14a-14e demonstrate distribution of HDAC2, HDAC3, HDAC6 and TPH2 mRNA and the effects of selected HDAC6 inhibitors on α-tubulin acetylation at lysine 40 (K40) and Histone H3 acetylation at lysine 9 (H3K9). FIG. 13f shows distribution of HDAC2, HDAC3, HDAC6 and TPH2 mRNA as seen after in situ hybridization on sagittal views of the mouse brain: the average expression intensities range from low (blue) to high (red): pictures are from Allen Institute for Brain Science. FIG. 14a shows that, in RN46A-B14 serotonergic cell line, the treatment for 4 h with the non-selective HDAC inhibitor trichostatin A (TSA, 0.6 μM) or the selective HDAC6 inhibitors tubastatin A (TubA, 2.5 μM), compound 73 (2.5 μM) and compound 101 (2.5 μM) all increased α-tubulin acetylation at K40. In contrast, only TSA significantly increased H3K9 acetylation in RN46A-B14 cells. Results are shown as percent change normalized to the vehicle treatment condition. The horizontal hard line depicts average and the dotted lines depict SEM of the vehicle treated wells. Representative Western blot images are presented. None of the treatments significantly altered total α-tubulin levels (n=3 per condition, middle row) or total levels of histone H3 protein. FIG. 14b shows that, in in vivo experiments, a single i.p. administration of compound 73 (5 mg/kg) and compound 101 (50 mg/kg) increased K40 acetylated α-tubulin levels measured at 30 min, 1 hour and 4 hour time-points in mouse whole brain lysates. In contrast, no significant changes were detected at any time-point after administration of tubastatin A (10 mg/kg), as expected based on the limited CNS bioavailability of this compound. Representative Western blot images are presented. FIG. 14c shows that the increases in α-tubulin K40 acetylation induced by administration of compound 73 (5 mg/kg) and compound 101 (50 mg/kg) were occluded in neuron-specific HDAC6 KO mice that had hyperacetylated α-tubulin at baseline. The drugs (5 mg/kg) were administered subchronically (at 24 h, 4 h and 30 min prior to sacrifice) to HDAC6 KO and WT littermates. The changes in tubulin acetylation were measured in tissue lysates from cortex (ctx), hippocampus (Hpc), dorsal Raphe nucleus (DRN) and cerebellum (cb). The tubulin acetylation levels in drug-treated mice are expressed as percent change of the vehicle-treated mice of the corresponding genotype. The horizontal lines depict the average tubulin acetylation±SEM in the vehicle treated mice. The representative Western blot images show the changes induced by each drug treatment in the hippocampus (left column) and dorsal raphe (right column) of WT and HDAC6 KO mice. Dramatically enhanced baseline levels of α-tubulin K40 acetylation in HDAC6 KO mice compared with WT mice treated with the vehicle were observed. No increase in KO mice treated with compound 73 and compound 101 in contrast to WT was observed. FIG. 14d shows that the Histone H3 lysine 9 (H3K9) acetylation measured by ChIP was increased at BDNF promoter 4 and cFOS promoters after the subchronic treatment with a high dose sodium butyrate (1.2 g/kg). FIG. 14e shows lack of effect of the subchronic treatment with a behaviorally active dose of compound 73 (5 mg/kg) on histone H3 lysine 9 (H3K9) acetylation at BDNF promoter 4 and cFOS promoters.

In contrast to the class I isoforms HDAC2 and 3, which are expressed highly and ubiquitously in the mouse brain, HDAC6 mRNA expression appears to be restricted to a small number of brain areas, with highest signal observed in DRN (FIG. 13f). In RN46A-B14 cells treated with TSA (0.6 μM), tubastatin A (2.5 μM), compound 73 (2.5 μM) or compound 101 (2.5 μM), Western blot analyses at 4 hours after the treatment revealed increases in the acetylated (lysine 40) fraction of α-tubulin (F_{4,9}=48.69, P<0.0001). No changes in total α-tubulin expression were detected (FIG. 14a). While TSA led to a 94% increase in acetylation of histone H3 at lysine 9 (F_{4,5}=22.21, P=0.0022), no significant change in histone H3K9 acetylation was observed following the treatment with compounds 73 or 101, or tubastatin A (FIG. 14a).

Although tubastatin A resulted in an increase in α-tubulin acetylation of about 400% in tissue culture (FIG. 14a), and produced a 268% increase of α-tubulin acetylation in heart (p<0.05) upon single systemic administration in vivo (10 mg/kg, 2.7 mM), it did not significantly change levels of α-tubulin acetylation in whole brain lysates (FIG. 14b). In contrast, compound 73 (5 mg/kg, 1.9 mM) and compound 101 (50 mg/kg, 15 mM) both led to significant increases in α-tubulin acetylation in whole brain lysates. The changes were significant at 30 minutes (F_{3,13}=163.4, P<0.0001), 1 hour (F_{3,13}=163.9, P<0.0001) and 4 hours (F_{3,14}=4.703, P=0.0179) after the administrations.

When compound 73 (5 mg/kg) or compound 101 (50 mg/kg) was administered repeatedly in wild-type mice at 24 h, 4 h and 30 m before sacrifice, significant increases in α-tubulin acetylation were observed in all tested brain regions (FIG. 14c): cortex, F_{2,7}=582.5, P<0.0001; hippocampus, F_{2,7}=260.4, P<0.0001; DRN, F_{2,7}=54.00, P<0.0001; and cerebellum, F_{2,7}=136.2, P<0.0001. In contrast, identical treatment regimens in KO mice did not produce increases in α-tubulin acetylation over baseline levels (FIG. 14c). Of note, as reported previously (Espallergues et al, 2012), these mutant mice dramatically enhanced the baseline acetylated α-tubulin (1.98±0.2 acetyl/total) when compared to their WT littermates (0.5±0.06; p<0.0001).

As described above in cell culture, treatment with compounds 73 and 101 in vivo did not significantly alter acetylation of histone 3 at lysine 9 in Western blot. Additionally, chromatin immunoprecipitation revealed no concurrent changes in histone H3K9 acetylation enrichment at promoter DNA within the hippocampus following the repeated i.p. treatment with 5 mg/kg compound 73. This is in contrast to the treatment with a behaviorally active dose of sodium butyrate (1.2 g/kg), a class I histone deacetylase inhibitor, which led to increases in enrichment of acetylated H3K9 at the promoter of activity-induced neuronal genes, namely cFOS and BDNF promoter 4 (FIGS. 14d and 14e).

### Antidepressant Effect of HDAC6 Inhibition

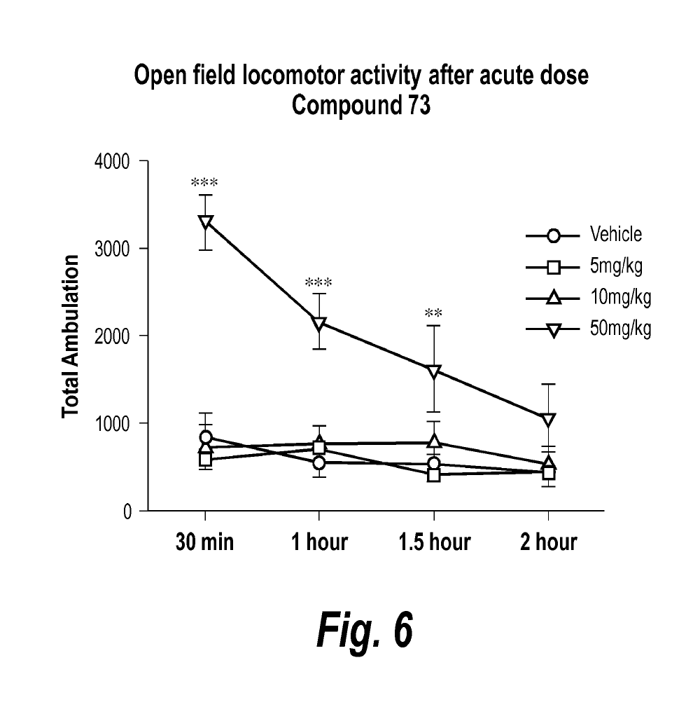

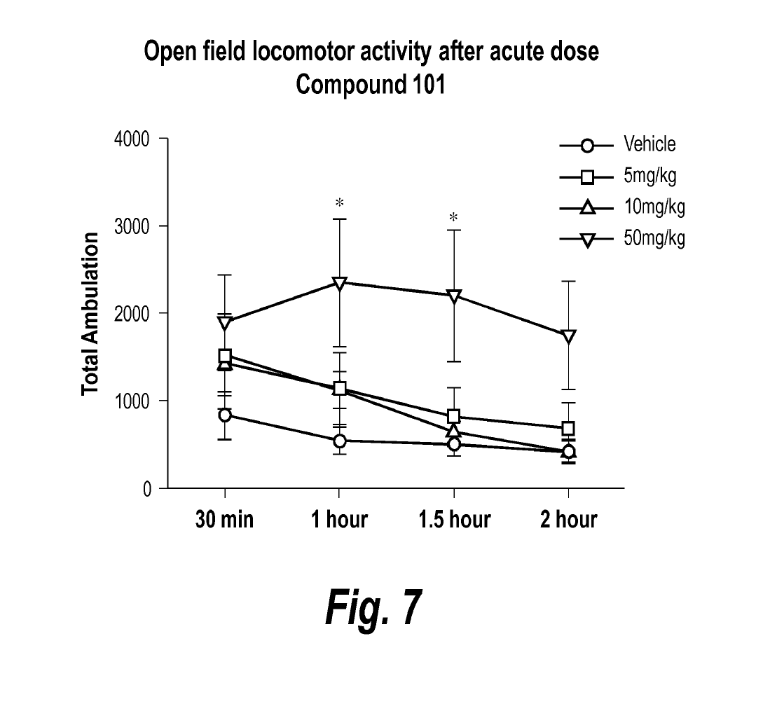

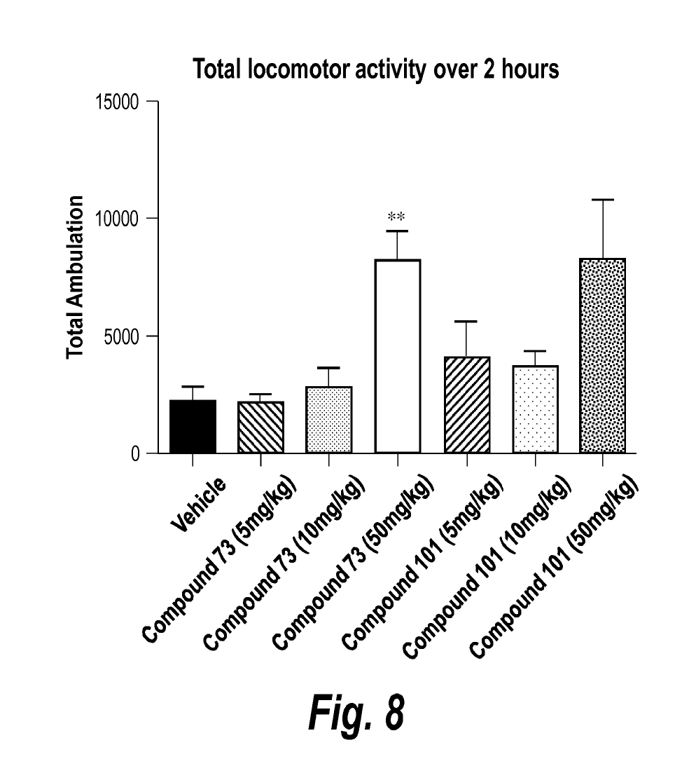

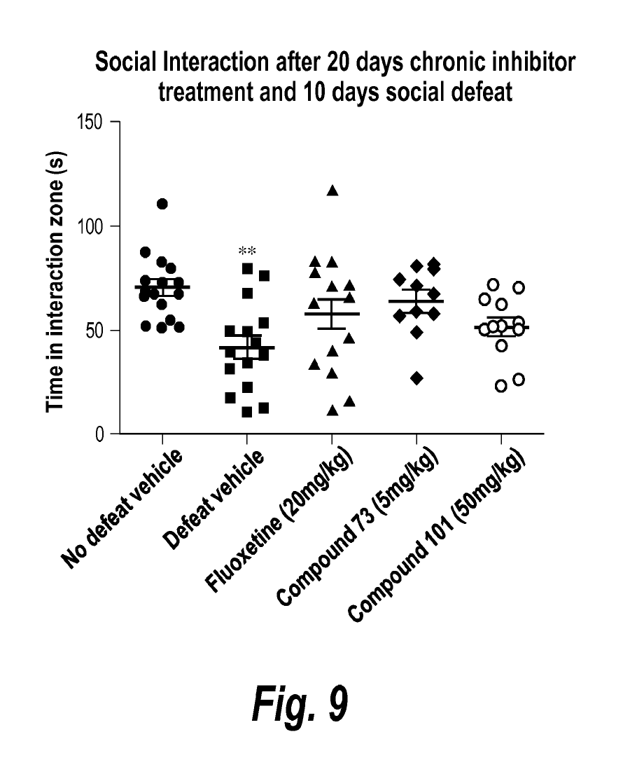

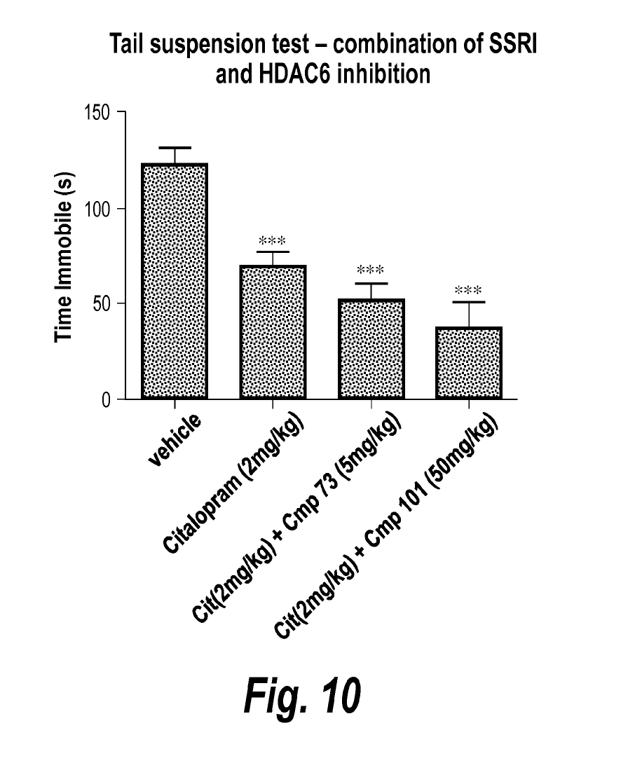

HDAC6 specific inhibitor compounds 73 and 101 have antidepressant-like activity. Compounds 73 and 101 both led to hyperlocomotor activity in the open field immediately after acute dose (See FIGS. 6, 7, and 8). Both compounds showed antidepressant activity comparable to the SSRI Citalopram in the tail suspension test 30 minutes after acute dose (See FIG. 11). Additionally, when combined with a dose of Citalopram with lower activity, these compounds seemed to have an additive effect (See FIG. 10). The dose of Citalopram chosen for this experiment was based on literature stating 2 mg/kg should be subactive, but this dose was found still showing an effect. Mice then underwent chronic social defeat stress, in which the mice are exposed to an aggressive resident mouse for 5 minutes daily for 10 days and are then tested on the 11th day in the social interaction test. Mice treated with vehicle for 20 total days, beginning 10 days before the defeat, show reduced time spent in the interaction zone when a target mouse is present when compared with control mice. Mice treated with HDAC6 inhibitor compounds showed resilience to developing this phenotype equivalent to that observed in mice treated with the SSRI Fluoxetine (See FIG. 9). The percent of mice found to be resilient, defined as spending more time in the interaction zone than the lowest control (i.e. ˜52 s), is higher in HDAC6 inhibitor groups. All behavior testing done using NIH Swiss mice, age 6-10 weeks. Tukey post-hoc analysis: * P=0.01 to 0.05, ** P=0.001-0.01, ***P<0.001 compared with vehicle treated mice.

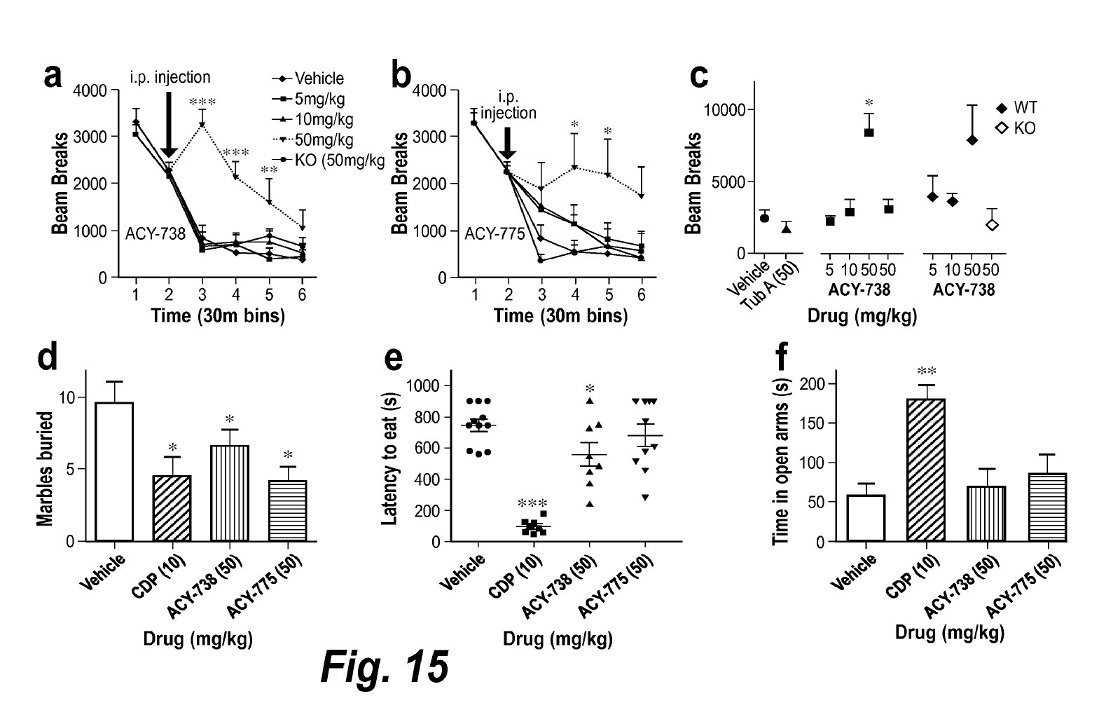

Data in FIGS. 15a-15f show the effects of HDAC6 selective inhibition in anxiety tests. As shown in FIGS. 15a, 15b and 15c, the activity was assessed in open-field test 1 h prior and 2 h after acute systemic administration of compound 73, compound 101 or tubastatin A. FIGS. 15a and 15b show that compound 73 and compound 101 increased exploration when administered at 50 mg/kg but not lower doses (arrow indicates drug administration), respectively. FIG. 15c shows that the beam breaks accumulated over 2 hours post treatment. Note the lack of effect of tubastatin A (10 mg/kg) and blockade of the effect of compound 73 and compound 101 in mice with neural-cell specific conditional HDAC6 KO (n=5-8 per condition). FIGS. 15d, 15e, and 15f show that the acute administration of compound 73 or compound 101 produced anxiolytic-like effects in marble burying test (FIG. 15d, n=10-18 per condition) and in NIH test (FIG. 15e, n=8-11 per condition), but had no effect in elevated plus maze (FIG. 15e, n=5 per condition).

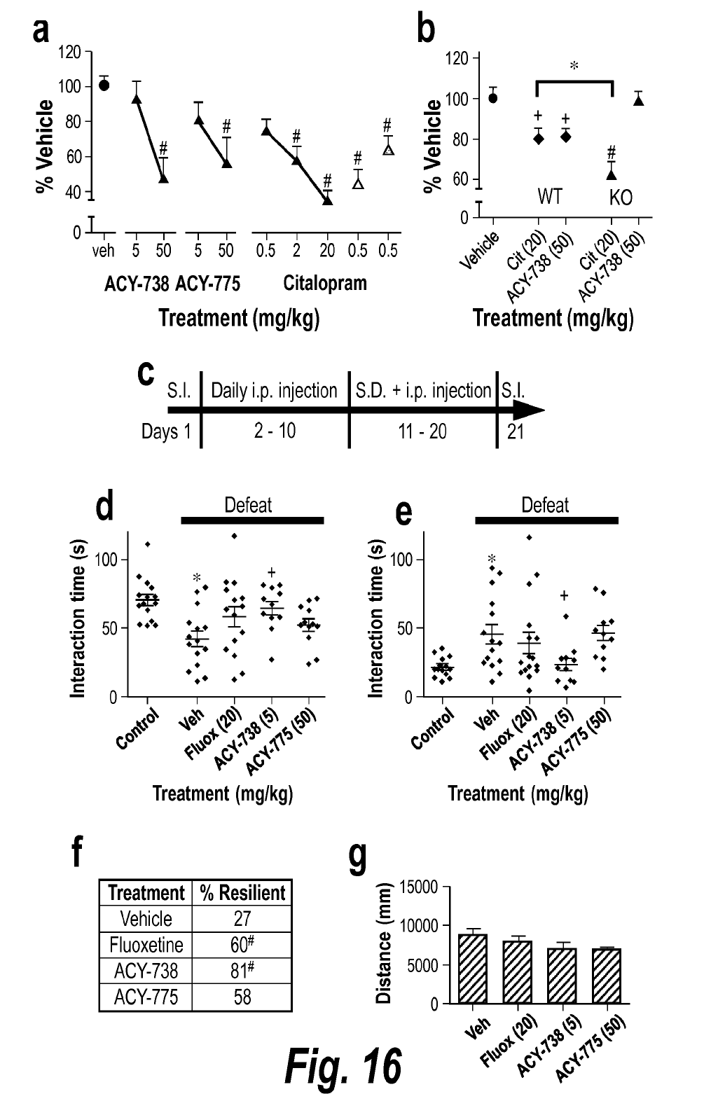

Data in FIGS. 16a-15g demonstrate that HDAC6 inhibitors compound 73 and compound 101 have antidepressant-like properties. FIG. 16a shows that, in the tail suspension test, acute administration of compound 73 and compound 101 produced anti-immobility effects in NIH Swiss mice resembling those of the reference antidepressant citalopram. The effects were observed 30 minutes after acute 50 mg/kg i.p. injection, but not for the lower dose (5 mg/kg). Combination of subactive doses of the HDAC6 selective inhibitors (5 mg/kg) and citalopram (0.5 mg/kg) produced robust potentiation of anti-immobility activity (n=8-29 per condition). FIG. 16b shows that, in C57BL/6J mice, the anti-immobility effect of compound 73 was blocked by a neural cell-selective KO of HDAC6, while citalopram effect was potentiated by HDAC6 KO. No difference was observed between HDAC6 WT and KO when treated with the vehicle (n=6-15 per condition). FIG. 16c is a timeline showing treatment period, social defeat and social interaction (SI) testing. Chronic treatment with the HDAC6-selective inhibitors prevented development of avoidance after social defeat as an increased average interaction time (FIG. 16d) and a decrease in time spent in the corners of the test arena (FIG. 16e) was observed, as compared to the vehicle treated mice that underwent social defeat. FIG. 16f shows that the treatment with SSRI or HDAC6 inhibitors results in a greater percentage of resilient mice compared to the treatment with the vehicle. FIG. 16g shows that no change in total distance traveled during the test period was observed (n=11-15 per condition).

Common antidepressants have been shown to increase ambulation in a novel environment (Brocco et al, 2002). The effects of acute administration of compound 73, compound 101 and tubastatin A on exploration in a novel open-field arena were tested. When injected with tubastatin A at 10 mg/kg after one hour of habituation, the mice did not exhibit changes of exploratory activity. However, rapid, dose-dependent hyperlocomotor effects were observed after the treatment with 50 mg/kg compound 73 (F_{4,132}=84.63, P<0.0001) (FIGS. 15a and 15c) or compound 101 (F_{4,174}=7.265, P<0.0001) (FIGS. 15b and 3c). The hyperlocomotor effects changed over time, (compound 73: F_{5,132}=84.63, P<0.0001; compound 101: F_{5,174}=28.71, P<0.001), peaking in the first hour after the injection and returning to the baseline by 2 hours, consistent with brain bio-distribution profiles. The same doses of compound 101 or compound 73 did not significantly alter open-field exploration in mice with neural cell selective KO HDAC6 (FIGS. 15a, 15b, and 15c). Distance in the center of the open field trended towards an increase, with compound 73 (602±96 beam breaks) and compound 101 (419±73 beam breaks) treated mice traveling a greater distance in the center than those treated with the vehicle (177±71 beam breaks). Furthermore, a 50 mg/kg dose of compound 73 failed to produce an enhancement of locomotor activity in WT mice tested in a home-cage environment (veh=4273±425 beam breaks; compound 73=3468±711 beam breaks; F_{1,232}=0.9447, P=0.36), suggesting that the effect reflects a disinhibition of exploratory behavior under neophobic condition, rather than a non-specific elevation of motor function.

In the marble-burying task, a test sensitive to both anxiolytics (Broekkamp et al, 1986) and antidepressants (Albelda and Joel, 2012), the treatment with compound 73 and compound 101 significantly reduced the number of marbles buried, to a similar degree as a non-sedating (10 mg/kg) dose of the common anxiolytic CDP (FIG. 15d; F_{3,50}=3.699, P=0.0176). Additionally, compound 73 given acutely at a dose of 50 mg/kg significantly decreased latency to eat in the NIH test (p<0.05), although this effect was not as potent as that of CDP (p<0.01) (FIG. 15e). In the EPM, a test sensitive to anxiolytics but not antidepressants, neither compound 73 nor compound 101 produced behavioral changes, while CDP dramatically increased the time in the open arms of the maze (FIG. 15f; F_{3,16}=7.498, P=0.0024).

In the TST (FIG. 16a), a significant effect of drug/dose was observed (F_{12,140}=9.368, P<0.0001). Significant reductions in immobility were detected at 50 mg/kg with either compound 73 (p<0.001) or compound 101 (p<0.01). No significant effect was observed after a 5 mg/kg dose of either drug. The administration of the SSRI citalopram led to significant decreases in time immobile at 2 mg/kg (p<0.001) and 20 mg/kg (p<0.001), but not at 0.5 mg/kg. At two hours after the injection, the treatment with 20 mg/kg citalopram (p<0.001) or 50 mg/kg compound 73 (p<0.01) still had significant activity.

To test whether anti-immobility effect of citalopram and compound 73 are dependent on intact HDAC6 protein levels, citalopram was administered to the mice with neural KO of HDAC6 or their WT littermates bred on a C57BL/6J background (FIG. 16b). In line with previous reports comparing C57BL/6J with NIH Swiss mice in TST (Lucid et al, 2001), it was found that, while the anti-immobility effects were still significant, the C57BL/6J WT had higher baseline immobility (p<0.0001) and were less responsive to both citalopram (p<0.0001) and compound 73 (p<0.01) than the NIH Swiss mice. In contrast, the anti-immobility effect of citalopram was significantly greater in HDAC6 KO mice than the WT littermates (F4,47=7.902, P<0.0001), in line with a previous report showing that the activity of another SSRI, fluoxetine, is amplified in HDAC6 KOs (Fukada et al, 2012). However, when anti-immobility activity of compound 73 was tested in HDAC6 KO mice, no significant effect was observed, indicating that an intact HDAC6 protein is required for compound 73 action in TST.

To further examine the interactions between SSRI and HDAC6, citalopram and the HDAC6 inhibitors were co-administered in NIH Swiss mice (FIG. 16a). Significant anti-immobility effects were observed for a sub-active dose of citalopram (0.5 mg/kg) co-administered with compound 73 or compound 101 (Cit+ compound 73 vs veh: p<0.001; Cit+ compound 101 vs veh: p<0.01) (FIG. 16a). The potency of the combination of citalopram with compound 73 was comparable to that of a 40 fold higher dose of citalopram.

A pro-resilient phenotype in the mouse CSD paradigm after serotonin-selective KO of HDAC6 was previously reported (Espallergues et al, 2012). Whether a chronic pharmacological inhibition of HDAC6 can replicate that phenotype is studied. Mice were pretreated for 10 days with either the vehicle, fluoxetine (20 mg/kg), compound 73 (5 mg/kg) or compound 101 (50 mg/kg), and were exposed to 10 days of CSD, with continued treatment (FIG. 16c). On day 21, 24 h after the last injection, the mice were tested in the social interaction test. Compared to the undefeated controls, the vehicle treated mice exposed to CSD showed a significant decrease in time in the interaction zone in the presence of a social target (FIG. 16d; p<0.01), indicating social avoidance. The treatments with fluoxetine, compound 73 or compound 101 prevented CSD-induced decrease in social interaction time. The defeated mice treated with compound 73 showed significantly higher time in the interaction zone than those treated with the vehicle (p<0.05). Similar trends were observed with compound 101 (p=0.18) and fluoxetine (p=0.08), albeit these effects did not reach post-hoc significance. Similarly, the treatment with compound 73 led to a decrease in the amount of time spent in the corner zones (FIG. 16e; p<0.05 vs vehicle). Significant effects were also observed when data was evaluated in terms of proportion of mice reaching criterion for resiliency. In the mice treated with the vehicle, 27% of mice were observed to be spontaneously resilient; the treatment with fluoxetine increased the percentage to 60% (p<0.05); the treatment with compound 73 increased the percentage to 81% (p<0.01); and the treatment with compound 101 increased the percentage to 58% (p=0.12)(FIG. 16F). The drug treatments did not change total distance traveled (FIG. 16g) or interaction time in undefeated control mice. There was no significant effect of compound 73 or compound 101 on body weight gain during the 4 weeks of treatment.

### CONCLUSIONS

Compounds 73 and 101, HDAC6 specific inhibitors are superior in selectivity for HDAC6 and brain bioavailability to the commercially available HDAC6 inhibitor, Tubastatin A.

These compounds produce significant increases in α-tubulin acetylation in cell culture and in the brain, without changing histone acetylation. Additionally, this effect is specific to HDAC6 inhibition, as it is blocked in HDAC6 knockout mice.

High doses of these compounds produce a hyperlocomotor/anxiolytic effect in the open field.

HDAC6 inhibitors show antidepressant-like effect in the tail suspension test as well as in a model of chronic social defeat stress.

This study shows that the use of brain penetrant, HDAC6-specific inhibitors in vivo show promise as a novel antidepressant-like drug.

### Example 9

### Pharmacokinetics and Blood-Brain Barrier Study

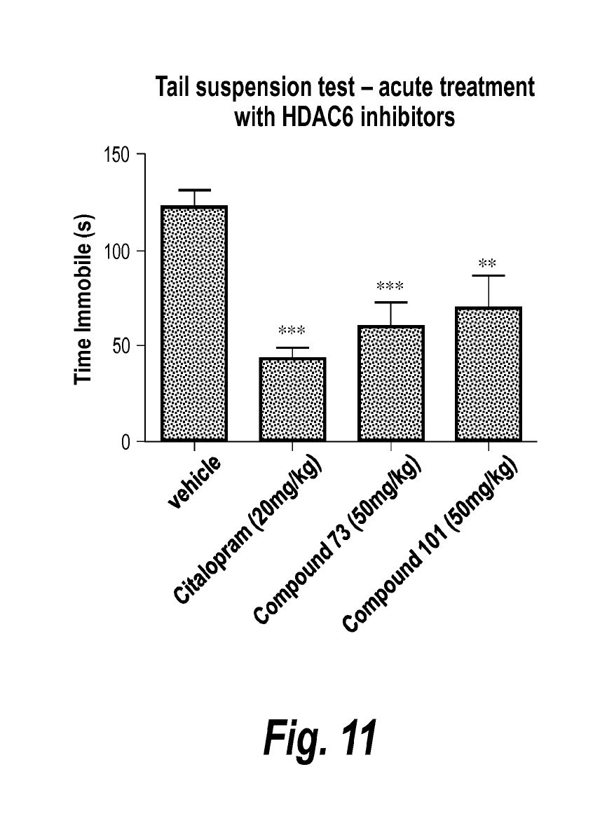

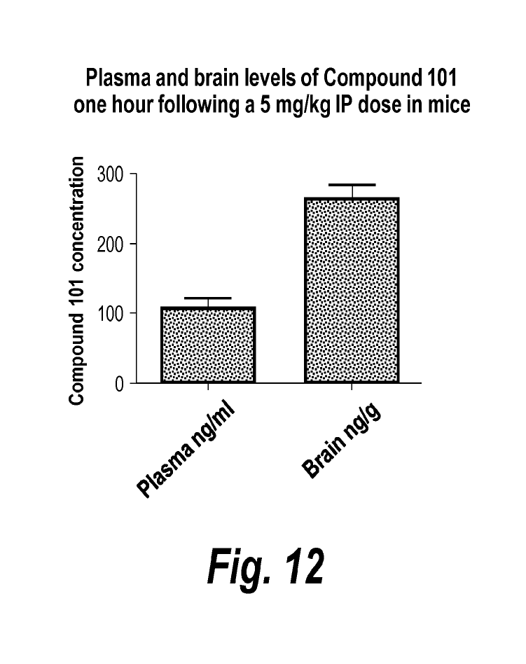

Male C57BL/6J mice were injected intraperitonealy with 5 mg/kg of compound 101 in a vehicle of 10% DMAC/10% solutol/80% saline Three mice per time point were sacrificed at predose, 5, 15, and 30 minutes, and 1, 2, 4, 8 and 24 hours. Plasma samples were taken from all mice upon sacrifice. Brain samples were taken from the mice sacrificed at 5 and 15 minutes and 1 and 4 hours. The brain samples were homogenized in PBS and centrifuged at 14000 rpm for 5 minutes. The supernatant from the brain homogenate and the plasma samples were injected onto a LC/MS/MS for analysis. The compound level in the samples was determined by a standard curve in the appropriate matrix. Pharmacokinetic parameters were determined from the raw data by WinNonLin. See FIG. 12. These new compounds exhibit better brain penetration with brain levels greater than or equal to levels seen in the blood plasma.

In addition, in vivo bio-distribution profiles of compounds 73 and 101, and tubastatin A were examined after acute dosing at 5 mg/kg or 50 mg/kg over 2 hours (data shown in Table 4). At t=30 minutes after the acute 50 mg/kg injection, respective plasma levels of compounds 73 and 101 were 515 ng/ml (1.9 μM) and 1359 ng/ml (4.1 μM). Elimination from plasma was rapid, with plasmatic half-life of 12 minutes and concentration below 10 ng/ml after two hours. Nevertheless, areas under concentration time curves for brain and plasma (AUC_{Brain}/AUCₚₗₐₛₘₐ), calculated over 2 hours for both compounds 73 and 101 led to ratios >1 (Table 4). In comparison, tubastatin A exhibited a longer plasmatic half-life of 2 hours, but a more limited brain penetration, with the AUC_{Brain}/AUCₚₗₐₛₘₐ ratio of 0.18. Taken together, these results suggest that despite their short half-life, compounds 73 and 101 rapidly distribute to the brain leading to a total drug exposure in CNS comparable to that of peripheral tissues.

TABLE 4

| | | | 30 min plasma | 30 min plasma | |
|---|---|---|---|---|---|
| | Cmax | T½ | concentration | concentration | |
| Com- | (ng/ | plasma | (ng/ml) - | (ng/ml) - | AUC_{Brain}/ |
| pound | ml) | (hrs) | 5 mg/kg | 50 mg/kg | AUCₚₗₐₛₘₐ |
| Tubastatin | 496 | 2 | 278 | N/A | 0.18 |
| A | | | | | |
| 73 | 494 | 0.2 | 89 | 515 | 1.22 |
| 101 | 915 | 0.5 | 496 | 1359 | 1.26 |

### INCORPORATION BY REFERENCE

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. A method for treating a subject suffering from or susceptible to depression and/or anxiety comprising administering to a subject in need thereof a therapeutically effective amount of a compound of formula I: 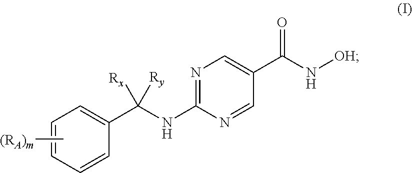
or a pharmaceutically acceptable salt, ester or prodrug thereof, wherein,
Rₓ and R_{y}, together with the carbon to which each is attached, form a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;
each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and
m is 0, 1, or 2;
to thereby treat the subject suffering from or susceptible to depression and/or anxiety.

2. The method of claim 1, wherein Rₓ and R_{y}, together with the carbon to which each is attached, form a cyclopropyl, cyclopentyl, cyclohexyl, or tetrahydropyran;

3. (canceled)

4. The method of claim 1, wherein m is 1 or 2, and each R_{A} is independently methyl, phenyl, F, Cl, methoxy, or CF₃.

5. (canceled)

6. The method of claim 1, wherein the compound of formula I is selected from the following: 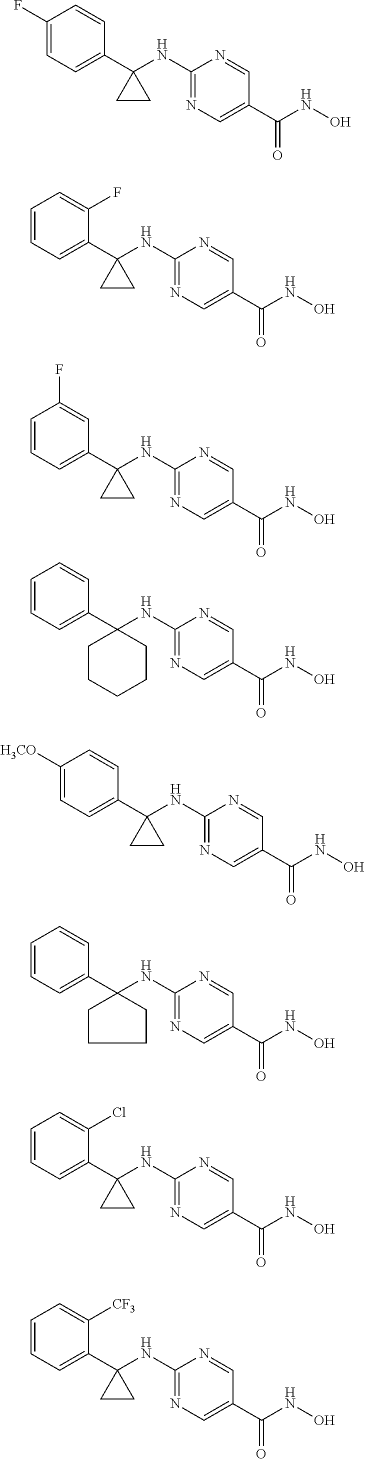 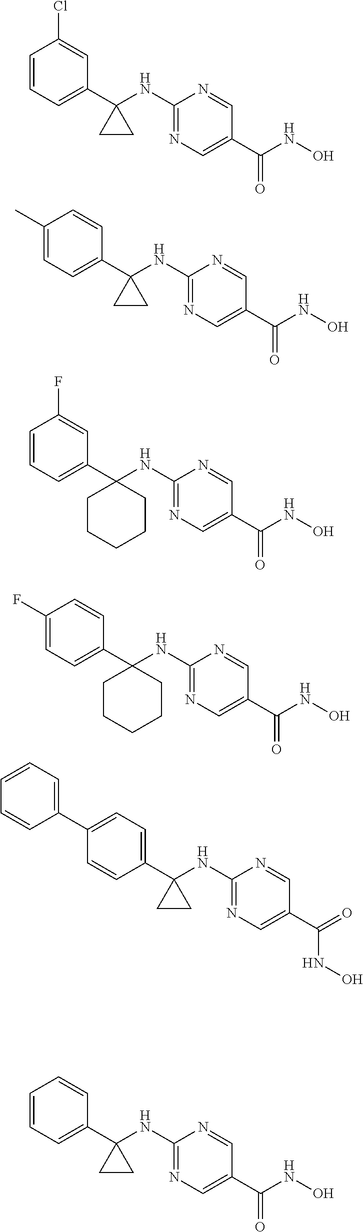 [SMILES: O=C(NO)c1cnc(NC2(c3cccc(Cl)c3)CC2)nc1 ; Cc1ccc(C2(Nc3ncc(C(=O)NO)cn3)CC2)cc1 ; O=C(NO)c1cnc(NC2(c3cccc(F)c3)CCCCC2)nc1 ; O=C(NO)c1cnc(NC2(c3ccc(F)cc3)CCCCC2)nc1 ; O=C(NO)c1cnc(NC2(c3ccc(-c4ccccc4)cc3)CC2)nc1 ; O=C(NO)c1cnc(NC2(c3ccccc3)CC2)nc1]
or a pharmaceutically acceptable salt, ester or prodrug thereof.

7. (canceled)

8. The method of claim 1, wherein the compound of formula I is:

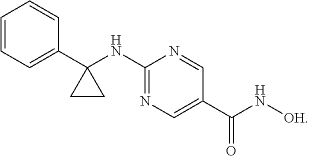 [SMILES: O=C(NO)c1cnc(NC2(c3ccccc3)CC2)nc1]

9. The method of claim 1, wherein the compound of formula I is:

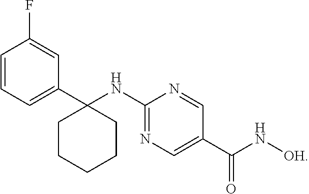 [SMILES: O=C(NO)c1cnc(NC2(c3cccc(F)c3)CCCCC2)nc1]

10. The method of claim 1, wherein the depression is selected from the group consisting of: depression, major depression, clinical depression, chronic depression, dysthymia, atypical depression, bipolar depression, manic depression, seasonal depression, phychotic depression, and postpartum depression.

11. The method of claim 1, wherein the anxiety is selected from the group consisting of: anxiety, generalized anxiety disorder, obsessive-compulsive disorder (OCD), panic disorder, post-traumatic stress disorder (PTSD), and social phobia (or social anxiety disorder).

12. The method of claim 1, wherein the disease is mediated by HDAC6.

13. The method of claim 1, wherein the compound of formula I penetrates the blood-brain barrier.

14. The method of claim 1, wherein the subject is a human.

15. A method for treating depression and/or anxiety comprising administering to a subject in need thereof a therapeutically effective amount of the following compound:

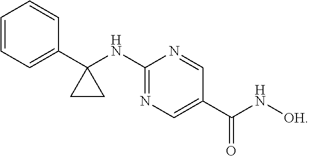 [SMILES: O=C(NO)c1cnc(NC2(c3ccccc3)CC2)nc1]

16. A method for treating depression and/or anxiety comprising administering to a subject in need thereof a therapeutically effective amount of the following compound:

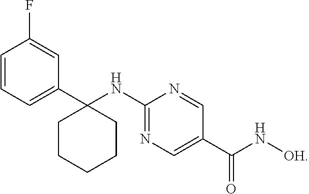 [SMILES: O=C(NO)c1cnc(NC2(c3cccc(F)c3)CCCCC2)nc1]

17. A kit comprising a compound of formula I: 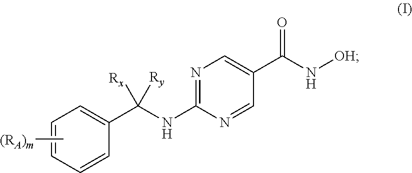
or a pharmaceutically acceptable salt, ester or prodrug thereof,
wherein,
Rₓ and R_{y}, together with the carbon to which each is attached, form a cyclopropyl, cyclobutyl, cyclopentyl, cyclohexyl, cycloheptyl, cyclooctyl, tetrahydropyran, piperidine, piperazine, morpholine, tetrahydrofuran, tetrahydrothiophene, pyrrolidine, oxozolidine, or imidazolidine, each of which is optionally substituted;
each R_{A} is independently alkyl, alkoxy, cycloalkyl, aryl, heterocycloalkyl, heteroaryl, arylalkyl, heteroarylalkyl, haloalkyl, haloalkoxy, halo, OH, —NO₂, —CN, or —NH₂; or two R_{A} groups together can form an optionally substituted cycloalkyl or heterocyclic ring; and
m is 0, 1, or 2;
and instructions for use in treating depression.

18. The method of claim 17, wherein Rₓ and R_{y}, together with the carbon to which each is attached, form a cyclopropyl, cyclopentyl, cyclohexyl, or tetrahydropyran;

19. (canceled)

20. The method of claim 17, wherein m is 1 or 2, and each R_{A} is independently methyl, phenyl, F, Cl, methoxy, or CF₃.

21. (canceled)

22. The method of claim 17, wherein the compound of formula I is:

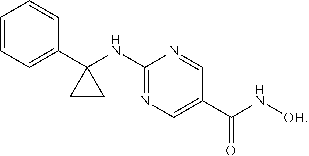 [SMILES: O=C(NO)c1cnc(NC2(c3ccccc3)CC2)nc1]

23. The method of claim 17, wherein the compound of formula I is:

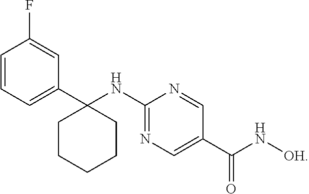 [SMILES: O=C(NO)c1cnc(NC2(c3cccc(F)c3)CCCCC2)nc1]

24. The method of claim 17, wherein the depression is selected from the group consisting of: major depression, clinical depression, chronic depression, dysthymia, atypical depression, bipolar depression, manic depression, seasonal depression, phychotic depression, and postpartum depression.

25. The method of claim 17, wherein the anxiety is selected from the group consisting of: generalized anxiety disorder, obsessive-compulsive disorder (OCD), panic disorder, post-traumatic stress disorder (PTSD), and social phobia (or social anxiety disorder).
